(19) United States
(12) Patent Application Publication
(10) Pub. No.: US 2024/0287102 A1
(43) Pub. Date: Aug. 29, 2024

(54) NOVEL CRYSTALLINE FORMS OF [(1R)-2-(1-BENZOFURAN-3-YL)-1-{[(1S,2R,4R)-7-OXABICYCLO[2.2.1]HEPTAN-2-YL]FORMAMIDO}ETHYL]BORONIC ACID, ADDUCTS THEREOF, AND PROCESSES TO OBTAIN

(71) Applicant: Merck Patent GmbH, Darmstadt (DE)

(72) Inventors: Delia-Maria GRUIA, Darmstadt (DE); Markus Klein, Darmstadt (DE); Michael Lange, Darmstadt (DE)

(73) Assignee: Merck Patent GmbH, Darmstadt (DE)

(21) Appl. No.: 18/291,703

(22) PCT Filed: Jul. 28, 2022
(86) PCT No.: PCT/EP2022/071167

§ 371 Date: Jan. 24, 2024

Related U.S. Application Data
(60) Provisional application No. 63/226,986, filed on Jul. 29, 2021.
(60) Provisional application No. 63/236,731, filed on Aug. 25, 2021.

(51) Int. Cl. C07F 5/02; A61K 31/69

(52) U.S. Cl. CPC C07F 5/025; A61K 31/69; C07B 2200/13

(57) ABSTRACT

A solid form of [(1R)-2-(1-benzofuran-3-yl)-1-{[(1S,2R,4R)-7-oxabicyclo[2.2.1]heptan-2-yl]formamido}ethyl]boronic acid, hydrates, solvates, and/or adducts thereof can be used as LMP7 inhibitors. The solid form can be a trimeric crystalline anhydrous form characterized by two or more 2θ XRPD peaks at 6.5±0.2°, 11.2±0.2°, 17.1±0.2°, 19.6±0.2°, and 21.9±0.2° degrees.

### TECHNICAL FIELD OF THE INVENTION

The present invention relates to solid forms of [(1R)-2-(1-benzofuran-3-yl)-1-{[(1S,2R,4R)-7-oxabicyclo[2.2.1]heptan-2-yl]formamido}ethyl]boronic acid (Compound 1) in substantially crystalline form or amorphous form, pharmaceutical compositions thereof, and methods of treatment therewith. The present invention relates to hydrates, solvates, esters and trimeric adducts of (Compound 1), as well as solid forms of said hydrates, solvates, esters and cyclotrimeric anhydrides, in substantially crystalline form, pharmaceutical compositions thereof, and methods of treatment therewith.

### BACKGROUND OF THE INVENTION

The proteasome is a high molecular weight, multisubunit protease which has been identified in every examined species from an archaebacterium to human. The enzyme has a native molecular weight of approximately 650,000 Da and, as revealed by electron microscopy, a distinctive cylinder-shaped morphology (Rivett, (1989) Arch. Biochem. Biophys. 268:1-8; and Orlowski, (1990) Biochemistry 29:10289-10297). The proteasome subunits range in molecular weight from 20,000 to 35,000, and are homologous to one another but not to any other known protease.

The 20S proteasome is a 700 kDa cylindrical-shaped multicatalytic protease complex comprised of 28 subunits, classified as α- and β-type, that are arranged in 4 stacked heptameric rings. In yeast and other eukaryotes, 7 different α subunits form the outer rings and 7 different β subunits comprise the inner rings. The α subunits serve as binding sites for the 19S (PA700) and 1 IS (PR68) regulatory complexes, as well as a physical barrier for the inner proteolytic chamber formed by the two β subunit rings. Thus, in vivo, the proteasome is believed to exist as a 26S particle (“the 26S proteasome”). In vivo experiments have shown that inhibition of the 20S form of the proteasome can be readily correlated to inhibition of 26S proteasome.

Cleavage of amino-terminal prosequences of β subunits during particle formation expose amino-terminal threonine residues, which serve as the catalytic nucleophiles. The subunits responsible for catalytic activity in proteasome thus possess an amino terminal nucleophilic residue, and these subunits belong to the family of N-terminal nucleophile (Ntn) ATTY REF: 26500-0023WO1 hydrolases (where the nucleophilic N-terminal residue is, for example, Cys, Ser, Thr, and other nucleophilic moieties). This family includes, for example, penicillin G acylase (PGA), penicillin V acylase (PVA), glutamine PRPP amidotransferase (GAT), and bacterial glycosylasparaginase. In addition to the ubiquitously expressed β subunits, higher vertebrates also possess three interferon-γ-inducible β subunits (LMP7, LMP2 and MECL1), which replace their normal counterparts, β5, β1 and β2, respectively. When all three IFN-γ-inducible subunits are present, the proteasome is referred to as an “immunoproteasome”. Thus, eukaryotic cells can possess two forms of proteasomes in varying ratios.

Through the use of different peptide substrates, three major proteolytic activities have been defined for the eukaryote 20S proteasomes: chymotrypsin-like activity (CT-L), which cleaves after large hydrophobic residues; trypsin-like activity (T-L), which cleaves after basic residues; and peptidylglutamyl peptide hydrolyzing activity (PGPH), which cleaves after acidic residues. Two additional less characterized activities have also been ascribed to the proteasome: BrAAP activity, which cleaves after branched-chain amino acids; and SNAAP activity, which cleaves after small neutral amino acids. Although both forms of the proteasome possess all five enzymatic activities, differences in the extent of the activities between the forms have been described based on specific substrates. For both forms of the proteasome, the major proteasome proteolytic activities appear to be contributed by different catalytic sites within the 20S core.

In eukaryotes, protein degradation is predominately mediated through the ubiquitin pathway in which proteins targeted for destruction are ligated to the 76 amino acid polypeptide ubiquitin. Once targeted, ubiquitinated proteins then serve as substrates for the 26S proteasome, which cleaves proteins into short peptides through the action of its three major proteolytic activities. While having a general function in intracellular protein turnover, proteasome-mediated degradation also plays a key role in many processes such as major histocompatibility complex (MHC) class I presentation, apoptosis and cell viability, antigen processing, NF-κB activation, and transduction of pro-inflammatory signals.

Proteasome activity is high in muscle wasting diseases that involve protein breakdown such as muscular dystrophy, cancer and AIDS. Evidence also suggests a possible role for the proteasome in the processing of antigens for the class I MHC molecules (Goldberg, et al. (1992) Nature 357:375-379).

Proteasomes are involved in neurodegenerative diseases and disorders such as Amyotrophic Lateral Sclerosis (ALS), (J Biol Chem 2003, Allen S et al., Exp Neurol 2005, Puttaparthi k et al.), Sjogren Syndrome (Arthritis & Rheumatism, 2006, Egerer T et al.), systemic lupus erythematoses and lupus nephritis (SLE/LN), (Arthritis & rheuma 2011, Ichikawa et al., J Immunol, 2010, Lang V R et al., Nat Med, 2008, Neubert K et al), glomerulonephritis (J Am Soc nephrol 2011, Bontscho et al.), Rheumatoid Arthritis (Clin Exp Rheumatol, 2009, Van der Heiden J W et al.), Inflammatory bowel disease (IBD), ulcerative colitis, crohn's diseases, (Gut 2010, Schmidt N et al., J Immunol 2010, Basler M et al., Clin Exp Immunol, 2009, Inoue S et al.), multiple sclerosis (Eur J Immunol 2008, Fissolo N et al., J Mol Med 2003, Elliott P J et al., J Neuroimmunol 2001, Hosseini et al., J Autoimmun 2000, Vanderlugt C L et al.), Amyotrophic lateral sclerosis (ALS), (Exp Neurol 2005, Puttaparthi k et al., J Biol Chem 2003, Allen S et al.), osteoarthritis (Pain 2011, Ahmed s et al., Biomed Mater Eng 2008, Etienne S et al.), Atherosclerosis (J Cardiovasc Pharmacol 2010, Feng B et al., Psoriasis (Genes & Immunity, 2007, Kramer U et al.), Myasthenia Gravis (J Immunol, 2011, Gomez A M et al.), Dermal fibrosis (Thorax 2011, Mutlu G M et al., Inflammation 2011, Koca S S et al., Faseb J 2006, Fineschi S et al.), renal fibrosis (Nephrology 2011 Sakairi T et al.), cardiac fibrosis (Biochem Pharmacol 2011, Ma y et al.), Liver fibrosis (Am J Physiol gastrointest Liver Physiol 2006, Anan A et al.), Lung fibrosis (Faseb J 2006, Fineschi S et al et al.), Imunoglobuline A nephropathy (IGa nephropathy), (Kidney Int, 2009, Coppo R et al.), Vasculitis (J Am Soc nephrol 2011, Bontscho et al.), Transplant rejection (Nephrol Dial transplant 2011, Waiser J et al.), Hematological malignancies (Br J Haematol 2011, singh A V et al., Curr Cancer Drug Target 2011, Chen D et al.) and asthma.

Yet, it should be noted that commercially available proteasome inhibitors inhibit both the constitutive and immuno-forms of the proteasome. Even bortezomib, the FDA-approved proteasome inhibitor for the treatment of relapsed multiple myeloma patients, does not distinguish between the two forms (Altun et al, Cancer Res 65:7896, 2005). Furthermore, the use of Bortezomib is associated with a treatment-emergent, painful peripheral neuropathy (PN), this bortezomib-induced neurodegeneration in vitro occurs via a proteasome-independent mechanism and that bortezomib inhibits several nonproteasomal targets in vitro and in vivo (Clin. Cancer Res, 17(9), May 1, 2011).

In addition to conventional proteasome inhibitors, a novel approach may be to specifically target the hematological-specific immunoproteasome, thereby increasing overall effectiveness and reducing negative off-target effects. It has been shown that immunoproteasome-specific inhibitor, could display enhanced efficiency on cells from a hematologic origin (Curr Cancer Drug Targets, 11(3), March, 2011).

Thus there is a need to provide new proteasome inhibitors that are selective of one specific form of the proteasome. In particular there is a need to provide selective immunoproteasome inhibitors, which could be used as therapeutic agents for the treatment of e.g. SLE or other immune or autoimmune disorders in the context of rheumatoid arthritis. Selective immunoproteasome inhibitors are helpful in order to minimize unwanted side effects mediated by inhibition of the constitutive proteasome or other nonproteasomal targets.

WO 2013/092979 A1 describes boronic acid derivatives, which show selectivity towards the inhibition of the LMP7 activity. However, the extent of selectivity, which is achievable with the described types of compounds, is limited, particularly with respect to the split to the inhibitory activity of the constitutive proteasome.

Unspecific inhibitors of the proteasome and the immunoproteasome like Bortezomib and Carfilzomib have demonstrated their clinical value in the indication of multiple myeloma. Although this unspecific profile, hitting major components in the immunoproteasome as well as the constitutive proteasome, is regarded beneficial in terms of target inhibition and clinical effectiveness, this unspecific profile limits the clinical applicability of these agents by inducing pronounced side effects like thrombocytopenia, neutropenia as well as peripheral neuropathy. To a certain extent, this side effect profile could be attributed to the broad inhibition of the catalytic activity, especially the combined inhibition of the β5 subunits of the constitutive and the immoproteasome. The approach to come up with more selective inhibitors of the immunoproteasome (and especially the β5i subunit of the immunoproteasome), in order to reduce major side effects has been described e.g. in 2011 by Singh et al (Br. J. Hematology 152(2): 155-163) for PR-924, a 100 fold selective inhibitor of the LMP7 subunit of the immunoproteasome. The authors demonstrated the presence of high expression levels of the immunoproteasome in multiple myeloma. The authors also described the effect of a selective inhibitor of the LMP7 subunit on the induction of cell death in MM cell lines as well as CD138+ MM primary patient cells without decreasing the viability of control PBMC's of healthy volunteers which can be regarded as a conceptual proof. Beside the concept of a reduced side effect profile for selective β5i inhibitors other group demonstrated the efficacy of selective β5i inhibition on the viability of Bortezomib resistant cell lines underlining the value and potential perspective for the application of selective LMP7 inhibitors for hematological malignancies (D. Niewerth et al./Biochemical Pharmacology 89 (2014) 43-51).

WO 2016/050356, WO 2016/050355, WO 2016/050359, and WO 2016/050358 describe compounds, which inhibit the activity of the immunoproteasome (LMP7) and provide a significant split to the inhibitory activity of the constitutive proteasome.

### SUMMARY OF THE INVENTION

It has now been found that solid forms of [(1R)-2-(1-benzofuran-3-yl)-1-{[(1S,2R,4R)-7-oxabicyclo[2.2.1]heptan-2-yl]formamido}ethyl]boronic acid (Compound 1), and pharmaceutically acceptable compositions thereof, are effective as inhibitors of LMP7.

In one aspect, Compound 1 is in a substantially anhydrous crystalline and salt free trimeric adduct form referred to as Form A1 as described and characterized herein. In a second aspect, Compound 1 is in a substantially anhydrous crystalline and salt free form which is a boronic ester formed with methanol and referred to as Form NF6 as described and characterized herein. In a third aspect, Compound 1 is in a substantially crystalline, salt free, mono-hydrated form referred to as Form NF2 as described and characterized herein. In a fourth aspect, Compound 1 is in a substantially crystalline, salt free and anhydrous form referred to as Form NF9 as described and characterized herein. In a fifth aspect, Compound 1 is in a substantially crystalline, salt free, boronic acid ester formed with isobutanol form referred to as Form NF3 as described and characterized herein. In a sixth aspect, Compound 1 is in a substantially crystalline, salt free, boronic acid ester formed with n-butanol form referred to as Form NF4 as described and characterized herein. In a seventh aspect, Compound 1 is in a substantially crystalline, salt free, boronic acid ester formed with 2-propanol form referred to as Form NF5 as described and characterized herein. In an eighth aspect, Compound 1 is in a substantially crystalline, salt free, hydrated trimeric boronic acid adduct with Pyridine referred to as Form NF7 as described and characterized herein. In a ninth aspect, Compound 1 is in a substantially crystalline, salt free, boronic acid ester formed with 1-propanol form referred to as Form NF8 as described and characterized herein.

The properties of a solid relevant to its efficacy as a drug can be dependent on the form of the solid. For example, in a drug substance, variation in the solid form can lead to differences in properties such as melting point, dissolution rate, oral absorption, bioavailability, toxicology results and clinical trial results.

Certain advantages of the following solid forms include the following:
Trimeric adduct form A1: crystalline morphic form with very good crystallinity; high thermal stability (mp>220° C.); slightly hygroscopic according to Ph. Eur. (section 5.11); high solubility in biorelevant media; and phase-pure manufacturability in large scale.
During the solution of the cyclotrimeric anhydride, addition of water leads to reformation of three molecules of cpd 1 in monomeric form.
The cyclotrimeric anhydride is more homogeneous compared to an amorphous powder, which may contain variable amounts of cyclic and acyclic oligomers.
Hydrate form NF2: crystalline morphic form with good crystallinity; high thermal stability (dehydration and phase transition to form A1>100° C.; mp>220° C.); slightly hygroscopic according to Ph. Eur. (section 5.11); highly soluble in biorelevant media; phase-pure manufacturability.
Anhydrous form NF9: crystalline morphic form with good crystallinity; high thermal stability (phase transition to form A1>150° C.; mp>220° C.); slightly hygroscopic according to Ph. Eur. (section 5.11).
Boronic acid ester form NF3: crystalline morphic form with good crystallinity; high thermal stability (mp/dec>150° C.).
Boronic acid ester form NF4: crystalline morphic form with good crystallinity; high thermal stability (mp/dec>130° C.).
Boronic acid ester form NF5: crystalline morphic form with good crystallinity; high thermal stability (dec>140° C.).
Boronic acid ester form NF6: crystalline morphic form with good crystallinity; high thermal stability (mp/dec>140° C.).
Hydrate of boronic acid trimeric adduct form NF7: crystalline morphic form with good crystallinity; high thermal stability (phase transition to form A1>100° C.; mp>220° C.); slightly hygroscopic according to Ph. Eur. (section 5.11).
Boronic acid ester form NF8: crystalline morphic form with good crystallinity; high thermal stability (phase transition to form A1>100° C.; mp>220° C.).

Solid forms of Compound 1, and pharmaceutically acceptable compositions thereof, are useful for treating a variety of diseases, disorders or conditions, associated with LMP7. Such diseases, disorders, or conditions include those described herein.

### BRIEF DESCRIPTION OF THE DRAWINGS

FIG. 1: Powder X-ray diffractogram of Form A1.

FIG. 2: DSC scan of free base form A1 (5 K/min).

FIG. 3: TGA scan of free base form A1 (5 K/min).

FIG. 4: Water Vapour Sorption Isotherm (25° C.) of free base form A1.

FIG. 5: Powder X-ray diffractogram of free base form NF2.

FIG. 6: DSC scan of form NF2 (5 K/min).

FIG. 7: TGA scan of form NF2 (5 K/min).

FIG. 8: Water Vapour Sorption Isotherm (25° C.) of form NF2.

FIG. 9: Powder X-ray diffractogram of anhydrous form NF9.

FIG. 10: DSC scan of anhydrous form NF9 (5 K/min).

FIG. 11: TGA scan of anhydrous form NF9 (5 K/min).

FIG. 12: Water Vapour Sorption Isotherm (25° C.) of anhydrous form NF9.

FIG. 13: Powder X-ray diffractogram of boronic acid ester form NF3.

FIG. 14: DSC scan of boronic acid ester form NF3 (5 K/min).

FIG. 15: TGA scan of boronic acid ester form NF3 (5 K/min).

FIG. 16: Powder X-ray diffractogram of boronic acid ester form NF4.

FIG. 17: DSC scan of boronic acid ester form NF4 (5 K/min).

FIG. 18: TGA scan of boronic acid ester form NF4 (5 K/min).

FIG. 19: Powder X-ray diffractogram of boronic acid ester form NF5.

FIG. 20: DSC scan of boronic acid ester form NF5 (5 K/min).

FIG. 21: TGA scan of boronic acid ester form NF5 (5 K/min).

FIG. 22: Powder X-ray diffractogram of boronic acid ester form NF6.

FIG. 23: DSC scan of boronic acid ester form NF6 (5 K/min).

FIG. 24: TGA scan of boronic acid ester form NF6 (5 K/min).

FIG. 25: Powder X-ray diffractogram of boronic acid ester form NF7.

FIG. 26: DSC scan of boronic acid ester form NF7 (5 K/min).

FIG. 27: TGA scan of boronic acid ester form NF7 (5 K/min).

FIG. 28: Water Vapour Sorption Isotherm (25° C.) of boronic acid ester form NF7.

FIG. 29: Powder X-ray diffractogram of boronic acid ester form NF8.

FIG. 30: DSC scan of boronic acid ester form NF8 (5 K/min).

FIG. 31: TGA scan of boronic acid ester form NF8 (5 K/min).

FIG. 32 shows the cyclic trimeric form of compound 1 that shows selective inhibition of the immunoproteasome subunit LMP7 (b5i), wherein, said compound comprises one embodiment of the present invention.

### DETAILED DESCRIPTION OF CERTAIN EMBODIMENTS

### 1. General Description of Compounds of the Invention

In certain aspects, the present invention provides for inhibitors of LMP7. In some embodiments, such compounds include those of the formulae described herein, or a pharmaceutically acceptable salt, hydrate, solvate or adduct thereof, wherein each variable is as defined and described herein.

### 2. Compounds and Definitions

As used herein the term “amorphous” refers to solid forms that consist of disordered arrangements of molecules and do not possess a distinguishable crystal lattice.

As used herein “crystalline” refers to compounds or compositions where the structural units are arranged in fixed geometric patterns or lattices, so that crystalline solids have rigid long range order. The structural units that constitute the crystal structure can be atoms, molecules, or ions. Crystalline solids show definite melting points.

The term “chemically stable”, as used herein, means that the solid form of Compound 1 does not decompose into one or more different chemical compounds when subjected to specified conditions, e.g., 40° C./75% relative humidity, for a specific period of time. e.g. 1 day, 2 days, 3 days, 1 week, 2 weeks, or longer. In some embodiments, less than 25% of the solid form of Compound 1 decomposes, in some embodiments, less than about 20%, less than about 15%, less than about 10%, less than about 5%, less than about 3%, less than about 1%, less than about 0.5% of the form of Compound 1 decomposes under the conditions specified. In some embodiments, no detectable amount of the solid form of Compound 1 decomposes.

The term “physically stable”, as used herein, means that the solid form of Compound 1 does not change into one or more different physical forms of Compound 1 (e.g. different solid forms as measured by XRPD, DSC, etc.) when subjected to specific conditions, e.g., 40° C./75% relative humidity, for a specific period of time. e.g. 1 day, 2 days, 3 days, 1 week, 2 weeks, or longer. In some embodiments, less than 25% of the solid form of Compound 1 changes into one or more different physical forms when subjected to specified conditions. In some embodiments, less than about 20%, less than about 15%, less than about 10%, less than about 5%, less than about 3%, less than about 1%, less than about 0.5% of the solid form of Compound 1 changes into one or more different physical forms of Compound 1 when subjected to specified conditions. In some embodiments, no detectable amount of the solid form of Compound 1 changes into one or more physically different solid forms of Compound 1.

As used herein, the phrase “substantially amorphous Compound 1” is used interchangeably with the phrases “amorphous Compound 1,” “amorphous Compound 1 substantially free of crystalline Compound 1,” and “substantially amorphous [(1R)-2-(1-benzofuran-3-yl)-1-{[(1S,2R,4R)-7-oxabicyclo[2.2.1]heptan-2-yl]formamido}ethyl]boronic acid.” In some embodiments, substantially amorphous Compound 1 has less than about 30% crystalline Compound 1, for example, less than about 30% of crystalline Compound 1, e.g., less than about 25% crystalline Compound 1, less than about 20% crystalline Compound 1, less than about 15% crystalline Compound 1, less than about 10% crystalline Compound 1, less than about 5% crystalline Compound 1, less than about 2% crystalline Compound 1.

As used herein, the phrase “substantially crystalline Compound 1” is used interchangeably with the phrases “Compound 1,” and “crystalline Compound 1 substantially free of amorphous Compound 1.” In some embodiments, substantially crystalline Compound 1 has less than about 30% amorphous Compound 1 or other solid forms, for example, less than about 30% of amorphous Compound 1 or other solid forms, e.g., less than about 25% amorphous Compound 1 or other solid forms, less than about 20% amorphous Compound 1 or other solid forms, less than about 15% amorphous Compound 1 or other solid forms, less than about 10% amorphous Compound 1 or other solid forms, less than about 5% amorphous Compound 1 or other solid forms, less than about 2% amorphous Compound 1 or other solid forms. In some embodiments, substantially crystalline Compound 1 has less than about 1% amorphous Compound 1 or other solid forms.

The term “substantially free” (as in the phrase “substantially free of form X”) when referring to a designated solid form of Compound 1 (e.g., an amorphous or crystalline form described herein) means that there is less than 20% (by weight) of the designated form(s) or co-form(s) (e.g., a crystalline or amorphous form of Compound 1) present, more preferably, there is less than 10% (by weight) of the designated form(s) present, more preferably, there is less than 5% (by weight) of the designated form(s) present, and most preferably, there is less than 1% (by weight) of the designated form(s) present.

The term “substantially pure” when referring to a designated solid form of Compound 1 (e.g., an amorphous or crystalline solid form described herein) means that the designated solid form contains less than 20% (by weight) of residual components such as alternate polymorphic or isomorphic crystalline form(s) or co-form(s) of Compound 1. It is preferred that a substantially pure solid form of Compound 1 contains less than 10% (by weight) of alternate polymorphic or isomorphic crystalline forms of Compound 1, more preferably less than 5% (by weight) of alternate polymorphic or isomorphic crystalline forms of Compound 1, and most preferably less than 1% (by weight) of alternate polymorphic or isomorphic crystalline forms of Compound 1.

As used herein, a “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase), or alternatively, an amorphous drug (dispersed phase) in an amorphous polymer (continuous phase). In some embodiments an amorphous solid dispersion includes the polymer constituting the dispersed phase, and the drug constitutes the continuous phase. In some embodiments, the dispersion includes amorphous Compound 1 or substantially amorphous Compound 1.

The term “solid amorphous dispersion” generally refers to a solid dispersion of two or more components, usually a drug and polymer, but possibly containing other components such as surfactants or other pharmaceutical excipients, where Compound 1 is amorphous or substantially amorphous (e.g., substantially free of crystalline Compound 1), and the physical stability and/or dissolution and/or solubility of the amorphous drug is enhanced by the other components.

As used herein, the terms “about” and “approximately”, when used in connection with doses, amounts, or weight percent of ingredients of a composition or a dosage form, mean a dose, amount, or weight percent that is recognized by one of ordinary skill in the art to provide a pharmacological effect equivalent to that obtained from the specified dose, amount, or weight percent. Specifically the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

The abbreviation “XRPD” stands for X-ray powder diffraction. The term XRPD is used interchangeably with PXRD.

The abbreviation “DSC” stands for differential scanning calorimetry.

The abbreviation “TGA” stands for thermogravimetric analysis.

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the term “modulator” is defined as a compound that binds to and/or inhibits the target with measurable affinity. In certain embodiments, a modulator has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in LMP7 activity between a sample comprising a compound of the present invention, or composition thereof, and LMP7, and an equivalent sample comprising LMP7, in the absence of said compound, or composition thereof.

### 3. Description of Exemplary Compounds

According to one aspect, the present invention provides a solid form of compound 1,

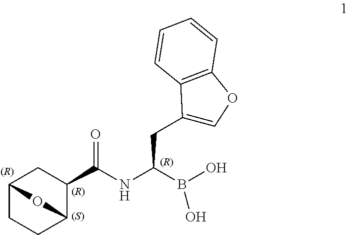

or a pharmaceutically acceptable salt thereof.

In certain aspects, the invention provides trimeric adduct form A1 of Compound 1, hydrate form NF2 of Compound 1, anhydrous form NF9 of Compound 1, boronic acid ester form NF3 of Compound 1, boronic acid ester form NF4 of Compound 1, boronic acid ester form NF5 of Compound 1, boronic acid ester form NF6 of Compound 1, hydrate of boronic acid trimeric adduct form NF7 of Compound 1, or a boronic acid ester form NF8 of Compound 1.

In one embodiment, the invention provides Compound 1 characterized as crystalline form A1.

In certain embodiments, form A1 is characterized by one or more 2θ peaks at 6.5 and 19.6 degrees. In certain embodiments, form A1 is characterized by one or more 2θ peaks at 6.5, 11.2, 17.1, 19.6, and 21.9 degrees. In certain embodiments, form A1 is characterized by two or more 2θ peaks at 6.5, 11.2, 17.1, 19.6, and 21.9 degrees. In certain embodiments, form A1 is characterized by three or more 2θ peaks at 6.5, 11.2, 17.1, 19.6, and 21.9 degrees. In certain embodiments, form A1 is characterized by four or more 2θ peaks at 6.5, 8.6, 11.2, 13.8, 14.1, 15.2, 16.3, 17.1, 18.2, 18.9, 19.6, 20.3, 20.7, 21.4, 21.9, 22.5 and 25.7 degrees. In certain embodiments, form A1 is characterized by 2θ peaks at 6.5, 11.2, 17.1, 19.6 and 20.7, degrees.

In certain embodiments, form A1 is characterized by 2θ peaks at

| | °2θ |
|---|---|
| | (Cu—Kα₁ |
| No. | radiation) ± 0.2° |
| 1 | 6.5 |
| 2 | 8.6 |
| 3 | 11.2 |
| 4 | 13.8 |
| 5 | 14.1 |
| 6 | 15.2 |
| 7 | 16.3 |
| 8 | 17.1 |
| 9 | 18.2 |
| 10 | 18.9 |
| 11 | 19.6 |
| 12 | 20.3 |
| 13 | 20.7 |
| 14 | 21.4 |
| 15 | 21.9 |
| 16 | 22.5 |
| 17 | 25.7 |

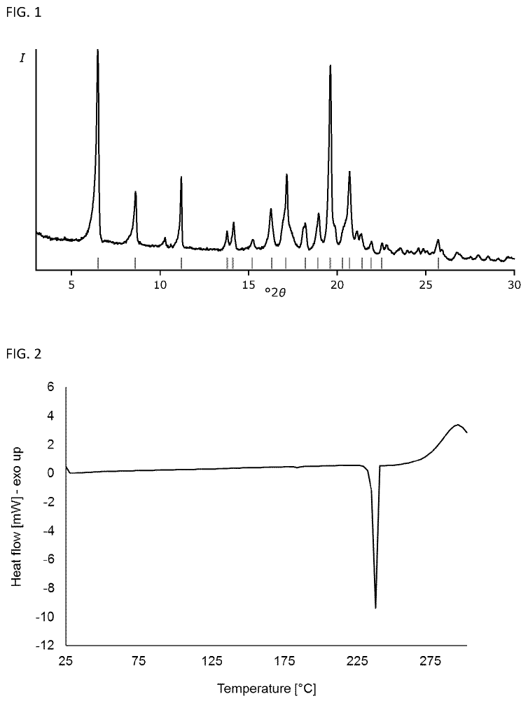

In another embodiment, form A1 is characterized by a diffraction pattern substantially similar to that of FIG. 1.

A Powder X-Ray Diffraction pattern of free base form A1 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In certain embodiments, form A1 is characterized by a crystal form, having a orthorhombic space group P-2₁2₁2₁ with the lattice parameters a=10.9863 Å, b=15.7954 Å, c=27.265 Å, and α=β=γ=90°. Reasonably rounded, the parameters are a=10.99±0.1 Å, b=15.80±0.1 Å, c=27.27±0.1 Å, and α=β=γ=90°. From the single crystal structure it is clear that form A1 represents a trimeric anhydrous form. Single crystal X-Ray Structure data were obtained on free base form A1 as well (SuperNova diffractometer from Agilent, equipped with CCD Detector using Cu K_{α} radiation at 298 K).

In certain embodiments, form A1 is an anhydrous form.

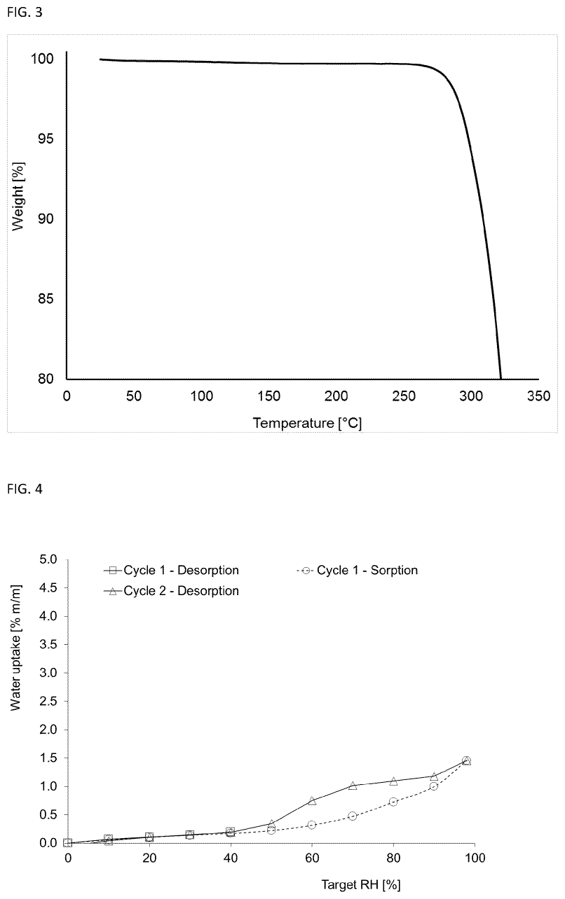

Other physical properties of trimeric form A1 include the following: Thermal behaviour of form A1 showed a melting peak onset greater than 220° C. Thermogravimetric analysis revealed a low weight loss of less than 2% m/m up to this temperature. DSC and TGA profiles are provided in FIGS. 2 and 3, respectively. DSC scan of trimeric form A1 was acquired on a Mettler-Toledo DSC 1 with a heating rate of 5 K/min, using nitrogen purge gas at 50 mL/min. TGA scan of trimeric form A1 was acquired on a Mettler-Toledo TGA 851 with a heating rate of 5 K/min, using nitrogen purge gas at 50 mL/min. Water Vapour Sorption behaviour of trimeric form A1 revealed small water uptake levels ≤1% m/m in the relative humidity (rh) range 0-80% rh, and very slightly elevated water uptake levels ≤2% m/m in the relative humidity (rh) range 90-98% rh. Trimeric form A1 can be classified as slightly hygroscopic according to Ph. Eur. Criteria (section 5.11.). Water Vapor Sorption isotherm (25° C.) of trimeric form A1 is provided in FIG. 4. Water Vapour Sorption isotherm was acquired on a DVS-Intrinsic system from SMS. Thermodynamic solubility (24 h) of trimeric form A1 at 37° C. was determined to be approximately 2.3 mg/mL in Simulated Gastric Fluid [SGF, pH 1.2], and approximately 2.0 mg/mL in USP Phosphate buffer [pH 7.4], respectively (see example 11). Dissolution level of trimeric form A1 in Simulated Gastric Fluid [SGF, pH 1.2] at 37° C. was determined to be approximately 2.4 mg/mL after 2 h (see example 12). Overall, trimeric form A1 revealed good solid-state properties (good crystallinity, slightly hygroscopic, high thermal stability, good solubility in biorelevant media) with good manufacturability in larger scale.

In one embodiment, the invention provides for Compound 1 characterized as hydrate crystalline form NF2.

In certain embodiments, hydrate form NF2 is characterized by one or more 2θ peaks at 7.0, 16.0, and 19.9 degrees. In certain embodiments, hydrate form NF2 is characterized by two or more 2θ peaks at 7.0, 16.0, and 19.9 degrees. In certain embodiments, hydrate form NF2 is characterized by 2θ peaks at 7.0, 16.0, and 19.9 degrees.

In certain embodiments, hydrate form NF2 is characterized by one or more 2θ peaks at 7.0, 16.0, 17.0, 18.1, 19.5, 19.9, 22.0, and 22.5 degrees. In certain embodiments, hydrate form NF2 is characterized by two or more 2θ peaks at 7.0, 16.0, 17.0, 18.1, 19.5, 19.9, 22.0, and 22.5 degrees. In certain embodiments, hydrate form NF2 is characterized by three or more 2θ peaks at 7.0, 16.0, 17.0, 18.1, 19.5, 19.9, 22.0, and 22.5 degrees. In certain embodiments, hydrate form NF2 is characterized by four or more 2θ peaks at 7.0, 16.0, 17.0, 18.1, 19.5, 19.9, 22.0, and 22.5 degrees. In certain embodiments, hydrate form NF2 is characterized by five or more 2θ peaks at 7.0, 16.0, 17.0, 18.1, 19.5, 19.9, 22.0, and 22.5 degrees. In certain embodiments, hydrate form NF2 is characterized by six or more 2θ peaks at 7.0, 16.0, 17.0, 18.1, 19.5, 19.9, 22.0, and 22.5 degrees. In certain embodiments, hydrate form NF2 is characterized by seven or more 2θ peaks at 7.0, 16.0, 17.0, 18.1, 19.5, 19.9, 22.0, and 22.5 degrees. In certain embodiments, hydrate form NF2 is characterized by 2θ peaks at 7.0, 16.0, 17.0, 18.1, 19.5, 19.9, 22.0, and 22.5 degrees.

In certain embodiments, hydrate form NF2 is characterized by 2θ peaks at

| | °2θ |
|---|---|
| | (Cu—Kα₁ |
| No. | radiation) ± 0.2° |
| 1 | 7.0 |
| 2 | 9.1 |
| 3 | 13.5 |
| 4 | 14.1 |
| 5 | 16.0 |
| 6 | 17.0 |
| 7 | 18.1 |
| 8 | 19.5 |
| 9 | 19.9 |
| 10 | 21.2 |
| 11 | 22.0 |
| 12 | 22.5 |
| 13 | 23.3 |
| 14 | 24.6 |
| 15 | 26.4 |
| 16 | 27.1 |
| 17 | 27.6 |
| 18 | 28.9 |

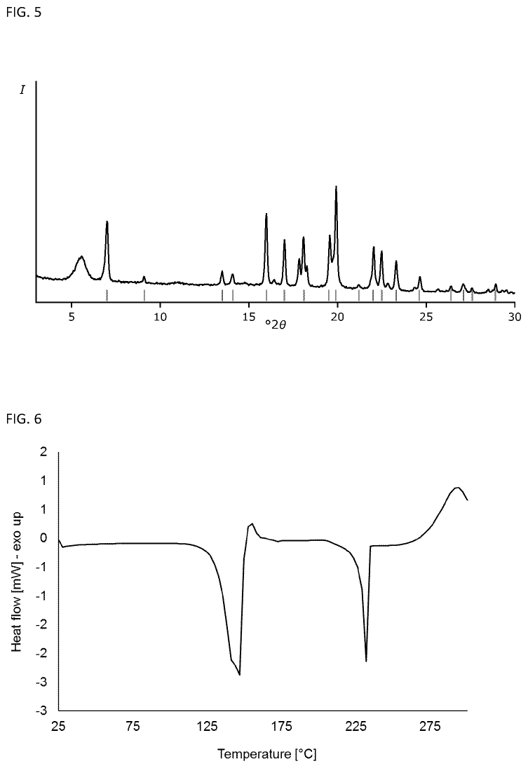

In another embodiment, hydrate form NF2 is characterized by a diffraction pattern substantially similar to that of FIG. 5.

A Powder X-Ray Diffraction pattern of hydrate form NF2 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In certain embodiments, hydrate form NF2 is a mono-hydrate form. In certain embodiments, free base hydrate form NF2 is a crystalline mono-hydrate form.

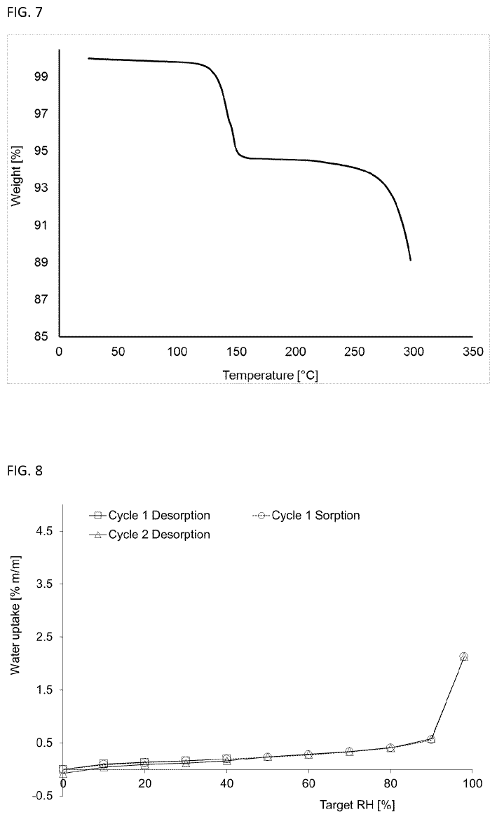

Other physical properties of hydrate form NF2 include the following: Thermal behaviour of hydrate form NF2 showed endothermic peaks greater than 100° C. followed by an exothermic phase transition to trimeric form A1 and finally the melting of the trimeric form A1 with a melting onset above 220° C. Thermogravimetric analysis revealed a weight loss step starting at greater than 100° C. of approximately 5-6% m/m. DSC and TGA profiles are displayed in FIGS. 6 and 7, respectively. DSC scan of hydrate form NF2 was acquired on a Mettler-Toledo DSC 1 with a heating rate of 5 K/min, using nitrogen purge gas at 50 mL/min. TGA scan of hydrate form NF2 was acquired on a Mettler-Toledo TGA 851 with a heating rate of 5 K/min, using nitrogen purge gas at 50 mL/min. Water Vapour Sorption behaviour of hydrate form NF2 revealed small water uptake levels ≤1% m/m in the relative humidity (rh) range 0-80% rh, and very slightly elevated water uptake levels ≤5% m/m in the relative humidity (rh) range 90-98% rh. Hydrate form NF2 can be classified as slightly hygroscopic according to Ph. Eur. Criteria (section 5.11.). Water Vapor Sorption isotherm (25° C.) of hydrate form NF2 is shown in FIG. 8. Water Vapour Sorption isotherm was acquired on a DVS-Intrinsic system from SMS. Thermodynamic solubility (24 h) of hydrate form NF2 at 37° C. was determined to be approximately 0.7 mg/mL in Simulated Gastric Fluid [SGF, pH 1.2], and approximately 0.7 mg/mL in Fasted-State Simulated Intestinal Fluid [FaSSIF, pH 6.5], respectively (see example 11). Overall, hydrate form NF2 revealed good solid-state properties (good crystallinity, slightly hygroscopic, high thermal stability, good solubility in biorelevant media).

In one embodiment, the invention provides for Compound 1 characterized as anhydrous form NF9.

In certain embodiments, anhydrous form NF9 is characterized by one or more 2θ peaks at 6.5, 18.0, 19.5, and 20.7 degrees. In certain embodiments, anhydrous form NF9 is characterized by two or more 2θ peaks at 6.5, 18.0, 19.5, and 20.7 degrees. In certain embodiments, anhydrous form NF9 is characterized by 2θ peaks at 6.5, 18.0, 19.5, and 20.7 degrees.

In certain embodiments, anhydrous form NF9 is characterized by one or more 2θ peaks at 6.5, 7.9, 16.2, 18.0, 19.5, and 20.7 degrees. In certain embodiments, anhydrous form NF9 is characterized by two or more 2θ peaks at 6.5, 7.9, 16.2, 18.0, 19.5, and 20.7 degrees. In certain embodiments, anhydrous form NF9 is characterized by three or more 2θ peaks at 6.5, 7.9, 16.2, 18.0, 19.5, and 20.7 degrees. In certain embodiments, anhydrous form NF9 is characterized by four or more 2θ peaks at 6.5, 7.9, 16.2, 18.0, 19.5, and 20.7 degrees. In certain embodiments, anhydrous form NF9 is characterized by five or more 2θ peaks at 6.5, 7.9, 16.2, 18.0, 19.5, and 20.7 degrees. In certain embodiments, anhydrous form NF9 is characterized by 2θ peaks at 6.5, 7.9, 16.2, 18.0, 19.5, and 20.7 degrees.

In certain embodiments, anhydrous form NF9 is characterized by 2θ peaks at

| | °2θ |
|---|---|
| | (Cu—Kα₁ |
| No. | radiation) ± 0.2° |
| 1 | 6.5 |
| 2 | 7.9 |
| 3 | 11.1 |
| 4 | 15.2 |
| 5 | 16.2 |
| 6 | 17.0 |
| 7 | 18.0 |
| 8 | 19.5 |
| 9 | 20.7 |
| 10 | 21.6 |

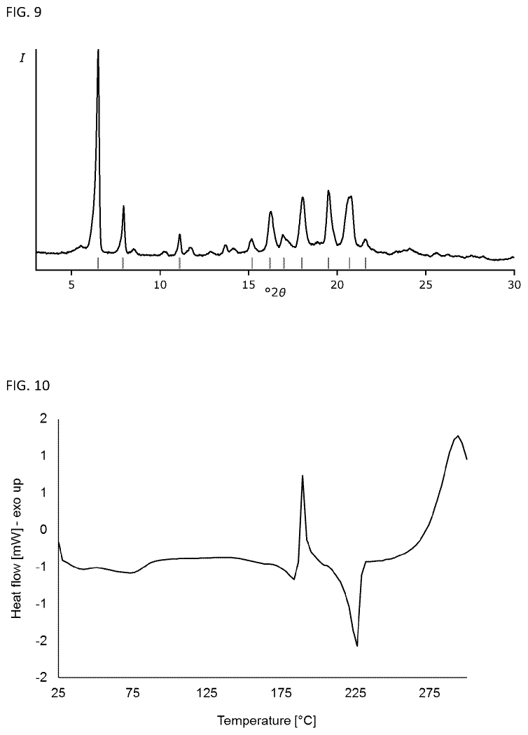

In another embodiment, anhydrous form NF9 is characterized by a diffraction pattern substantially similar to that of FIG. 9.

A Powder X-Ray Diffraction pattern of anhydrous form NF9 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In another embodiment, form NF9 is an anhydrous form.

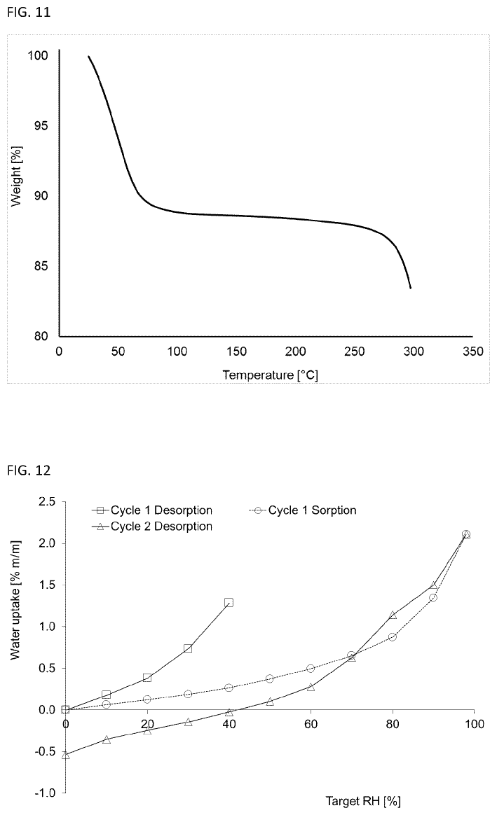

Other physical properties of anhydrous form NF9 include the following: Thermal behaviour of anhydrous form NF9 showed broad endothermic peaks in the DSC up to 100° C. accompanied by a weight loss step in the TGA. In the DSC, a phase transition to trimeric form A1 occurred above 150° C., and the trimeric A1 form has a melting peak onset greater than 220° C. DSC and TGA profiles are displayed in FIGS. 10 and 11, respectively. DSC scan of the anhydrous form NF9 was acquired on a Mettler-Toledo DSC 1 with a heating rate of 5 K/min, using nitrogen purge gas at 50 mL/min. TGA scan of anhydrous form NF9 was acquired on a Mettler-Toledo TGA 851 with a heating rate of 5 K/min, using nitrogen purge gas at 50 mL/min. Water Vapour Sorption behaviour of anhydrous form NF9 revealed small water uptake levels ≤1% m/m in the relative humidity (rh) range 0-80% rh, and slightly elevated water uptake levels ≤5% m/m in the relative humidity (rh) range 90-98% rh. Anhydrous form NF9 can be classified as slightly hygroscopic according to Ph. Eur. Criteria (section 5.11.). Water Vapor Sorption isotherm (25° C.) of anhydrous form NF9 is displayed in FIG. 12. Water Vapour Sorption isotherm was acquired on a DVS-Intrinsic system from SMS. Overall, anhydrous form NF9 revealed good solid-state properties (good crystallinity, slightly hygroscopic, high thermal stability).

In one embodiment, the invention provides for Compound 1 characterized as boronic acid ester form NF3.

In certain embodiments, boronic acid ester form NF3 is characterized by one or more 2θ peaks at 7.8, 17.1, 20.6, 21.2, and 22.0 degrees. In certain embodiments, boronic acid ester form NF3 is characterized by two or more 2θ peaks at 7.8, 17.1, 20.6, 21.2, and 22.0 degrees. In certain embodiments, boronic acid ester form NF3 is characterized by 2θ peaks at 7.8, 17.1, 20.6, 21.2, and 22.0 degrees.

In certain embodiments, boronic acid ester form NF3 is characterized by one or more 2θ peaks at 7.8, 12.5, 17.1, 20.6, 21.2, and 22.0 degrees. In certain embodiments, boronic acid ester form NF3 is characterized by two or more 2θ peaks at 7.8, 12.5, 17.1, 20.6, 21.2, and 22.0 degrees. In certain embodiments, boronic acid ester form NF3 is characterized by three or more 2θ peaks at 7.8, 12.5, 17.1, 20.6, 21.2, and 22.0 degrees. In certain embodiments, boronic acid ester form NF3 is characterized by four or more 2θ peaks at 7.8, 12.5, 17.1, 20.6, 21.2, and 22.0 degrees. In certain embodiments, boronic acid ester form NF3 is characterized by 2θ peaks at 7.8, 12.5, 17.1, 20.6, 21.2, and 22.0 degrees.

In certain embodiments, boronic acid ester form NF3 is characterized by 2θ peaks at

| | °2θ |
|---|---|
| | (Cu—Kα₁ |
| No. | radiation) ± 0.2° |
| 1 | 7.8 |
| 2 | 12.5 |
| 3 | 17.1 |
| 4 | 18.6 |
| 5 | 20.6 |
| 6 | 21.2 |
| 7 | 22.0 |
| 8 | 24.7 |

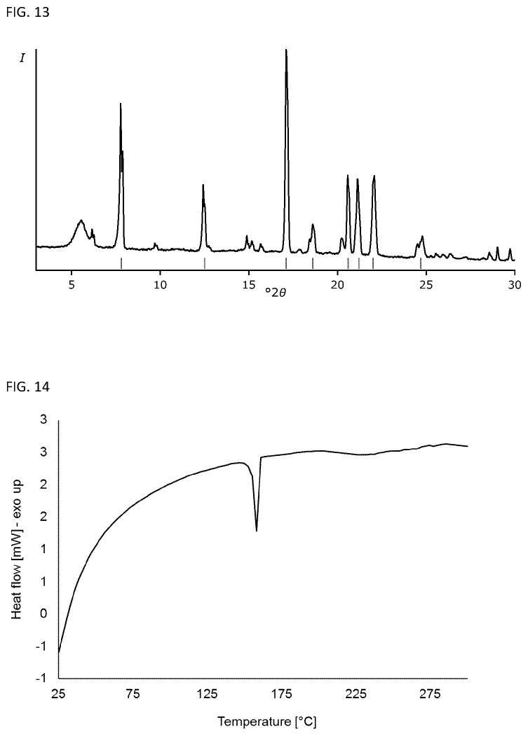

In another embodiment, boronic acid ester form NF3 is characterized by a diffraction pattern substantially similar to that of FIG. 13.

A Powder X-Ray Diffraction pattern of boronic acid ester form NF3 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In another embodiment, boronic acid ester form NF3 is characterized as a crystalline anhydrous form.

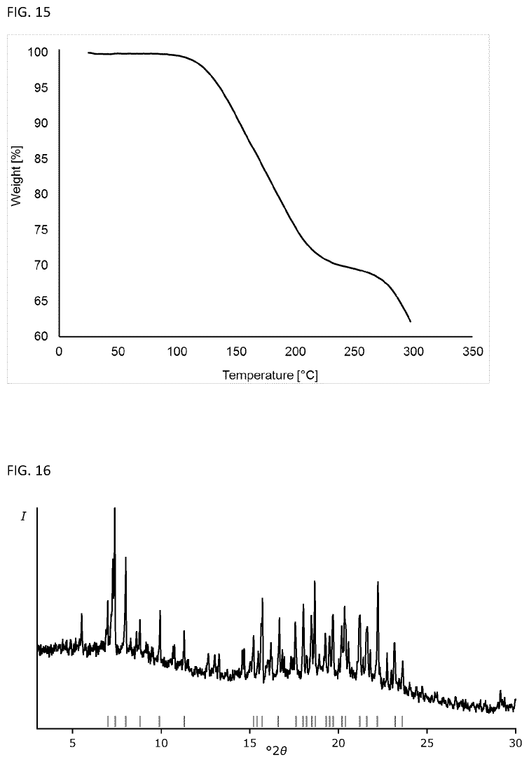

Other physical properties of boronic acid ester form NF3 include the following: Thermal behaviour of boronic acid ester form NF3 showed an endothermic melting/decomposition peak in the DSC at temperatures greater than 150° C. accompanied by a weight loss step in the TGA. DSC and TGA profiles are displayed in FIGS. 14 and 15, respectively. DSC scan of the boronic acid ester form NF3 was acquired on a Mettler-Toledo DSC 1 with a heating rate of 5 K/min, using nitrogen purge gas at 50 mL/min. TGA scan of boronic acid ester form NF3 was acquired on a Mettler-Toledo TGA 851 with a heating rate of 5 K/min, using nitrogen purge gas at 50 mL/min. Overall, boronic acid ester form NF3 reveals good solid-state properties (good crystallinity, high thermal stability).

In one embodiment, the invention provides for Compound 1 characterized as boronic acid ester form NF4.

In certain embodiments, boronic acid ester form NF4 is characterized by one or more 2θ peaks at 7.4, 8.0, 18.0, 18.7, and 22.2 degrees. In certain embodiments, boronic acid ester form NF4 is characterized by two or more 2θ peaks at 7.4, 8.0, 18.0, 18.7, and 22.2 degrees. In certain embodiments, boronic acid ester form NF4 is characterized by 2θ peaks at 7.4, 8.0, 18.0, 18.7, and 22.2 degrees.

In certain embodiments, boronic acid ester form NF4 is characterized by one or more 2θ peaks at 7.4, 8.0, 15.4, 16.6, 18.0, 18.5, 18.7, 20.2 and 22.2 degrees. In certain embodiments, boronic acid ester form NF4 is characterized by two or more 2θ peaks at 7.4, 8.0, 15.4, 16.6, 18.0, 18.5, 18.7, 20.2 and 22.2 degrees. In certain embodiments, boronic acid ester form NF4 is characterized by three or more 2θ peaks at 7.4, 8.0, 15.4, 16.6, 18.0, 18.5, 18.7, 20.2 and 22.2 degrees. In certain embodiments, boronic acid ester form NF4 is characterized by four or more 2θ peaks at 7.4, 8.0, 15.4, 16.6, 18.0, 18.5, 18.7, 20.2 and 22.2 degrees. In certain embodiments, boronic acid ester form NF4 is characterized by five or more 2θ peaks at 7.4, 8.0, 15.4, 16.6, 18.0, 18.5, 18.7, 20.2 and 22.2 degrees. In certain embodiments, boronic acid ester form NF4 is characterized by six or more 2θ peaks at 7.4, 8.0, 15.4, 16.6, 18.0, 18.5, 18.7, 20.2 and 22.2 degrees. In certain embodiments, boronic acid ester form NF4 is characterized by 2θ peaks at 7.4, 8.0, 15.4, 16.6, 18.0, 18.5, 18.7, 20.2 and 22.2 degrees.

In certain embodiments, boronic acid ester form NF4 is characterized by 2θ peaks at

| | °2θ |
|---|---|
| | (Cu—Kα₁ |
| No. | radiation) ± 0.2° |
| 1 | 7.0 |
| 2 | 7.4 |
| 3 | 8.0 |
| 4 | 8.8 |
| 5 | 9.9 |
| 6 | 11.3 |
| 7 | 15.2 |
| 8 | 15.4 |
| 9 | 15.7 |
| 10 | 16.6 |
| 11 | 17.6 |
| 12 | 18.0 |
| 13 | 18.2 |
| 14 | 18.5 |
| 15 | 18.7 |
| 16 | 19.3 |
| 17 | 19.5 |
| 18 | 19.7 |
| 19 | 20.2 |
| 20 | 20.4 |
| 21 | 21.2 |
| 22 | 21.6 |
| 23 | 22.2 |
| 24 | 23.2 |
| 25 | 23.6 |

In another embodiment, boronic acid ester form NF4 is characterized by a diffraction pattern substantially similar to that of FIG. 16.

A Powder X-Ray Diffraction pattern of boronic acid ester form NF4 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In another embodiment, boronic acid ester form NF4 is characterized as a crystalline form.

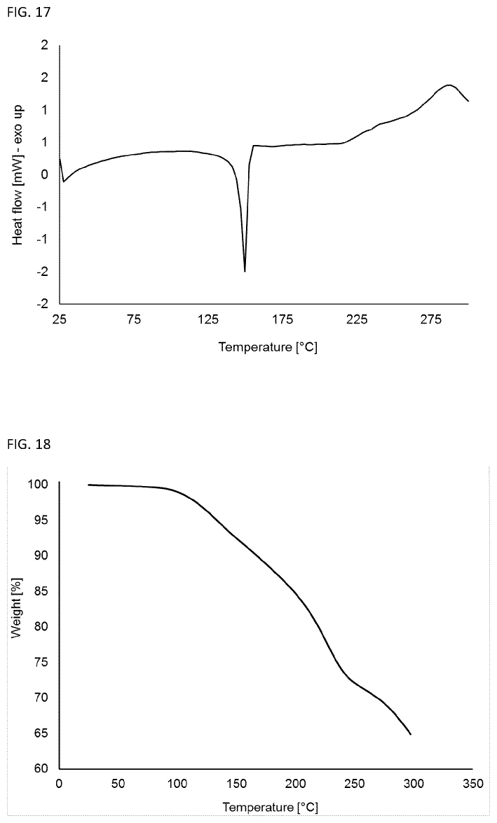

Other physical properties of boronic acid ester form NF4 include the following: Thermal behaviour of boronic acid ester form NF4 showed an endothermic melting/decomposition peak in the DSC at temperatures greater than 130° C. accompanied by a weight loss step in the TGA. DSC and TGA profiles are displayed in the FIGS. 17 and 18, respectively. DSC scan of boronic acid ester form NF4 was acquired on a Mettler-Toledo DSC 1 with a heating rate of 5 K/min, using nitrogen purge gas at 50 mL/min. TGA scan of boronic acid ester form NF4 was acquired on a Mettler-Toledo TGA 851 with a heating rate of 5 K/min, using nitrogen purge gas at 50 mL/min. Overall, boronic acid ester form NF4 revealed good solid-state properties (good crystallinity, high thermal stability).

In one embodiment, the invention provides for Compound 1 characterized as boronic acid ester form NF5.

In certain embodiments, boronic acid ester form NF5 is characterized by one or more 2θ peaks at 5.8, 18.4, 18.7, 19.0 and 21.7 degrees. In certain embodiments, boronic acid ester form NF5 is characterized by two or more 2θ peaks at 5.8, 18.4, 18.7, 19.0 and 21.7 degrees. In certain embodiments, boronic acid ester form NF5 is characterized by 2θ peaks 5.8, 18.4, 18.7, 19.0 and 21.7 degrees.

In certain embodiments, boronic acid ester form NF5 is characterized by one or more 2θ peaks at 5.8, 12.8, 17.6, 18.4, 18.7, 19.0, 20.1 and 21.7 degrees. In certain embodiments, boronic acid ester form NF5 is characterized by two or more 2θ peaks at 5.8, 12.8, 17.6, 18.4, 18.7, 19.0, 20.1 and 21.7 degrees. In certain embodiments, boronic acid ester form NF5 is characterized by three or more 2θ peaks at 5.8, 12.8, 17.6, 18.4, 18.7, 19.0, 20.1 and 21.7 degrees. In certain embodiments, boronic acid ester form NF5 is characterized by four or more 2θ peaks at 5.8, 12.8, 17.6, 18.4, 18.7, 19.0, 20.1 and 21.7 degrees. In certain embodiments, boronic acid ester form NF5 is characterized by five or more 2θ peaks at 5.8, 12.8, 17.6, 18.4, 18.7, 19.0, 20.1 and 21.7 degrees. In certain embodiments, boronic acid ester form NF5 is characterized by six or more 2θ peaks at 5.8, 12.8, 17.6, 18.4, 18.7, 19.0, 20.1 and 21.7 degrees. In certain embodiments, boronic acid ester form NF5 is characterized by seven or more 2θ peaks at 5.8, 12.8, 17.6, 18.4, 18.7, 19.0, 20.1 and 21.7 degrees. In certain embodiments, boronic acid ester form NF5 is characterized by 2θ peaks at 5.8, 12.8, 17.6, 18.4, 18.7, 19.0, 20.1 and 21.7 degrees.

In certain embodiments, boronic acid ester form NF5 is characterized by 2θ peaks at

| | °2θ |
|---|---|
| | (Cu—Kα₁ |
| No. | radiation) ± 0.2° |
| 1 | 5.8 |
| 2 | 11.7 |
| 3 | 12.4 |
| 4 | 12.8 |
| 5 | 14.9 |
| 6 | 15.6 |
| 7 | 16.2 |
| 8 | 17.6 |
| 9 | 18.4 |
| 10 | 18.7 |
| 11 | 19.0 |
| 12 | 20.1 |
| 13 | 20.5 |
| 14 | 21.0 |
| 15 | 21.7 |
| 16 | 22.4 |
| 17 | 23.2 |
| 18 | 24.5 |
| 19 | 24.9 |
| 20 | 25.3 |
| 21 | 26.0 |
| 22 | 26.6 |

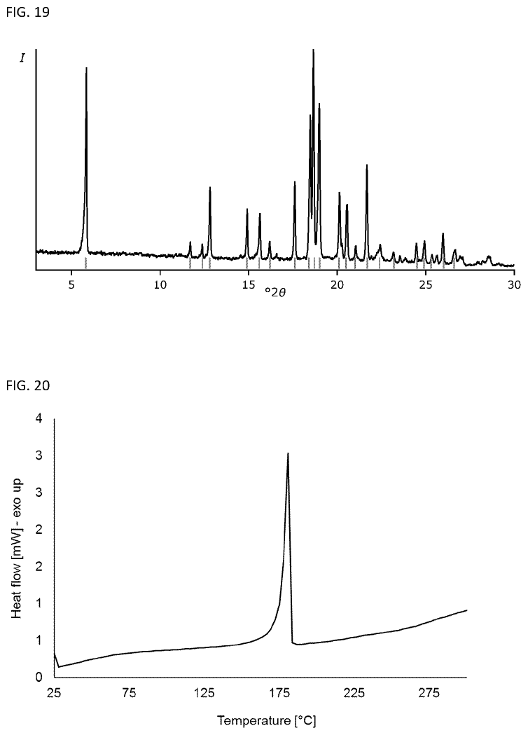

In another embodiment, boronic acid ester form NF5 is characterized by a diffraction pattern substantially similar to that of FIG. 19.

A Powder X-Ray Diffraction pattern of free base boronic acid ester form NF5 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In another embodiment, boronic acid ester form NF5 is characterized as a crystalline anhydrous form.

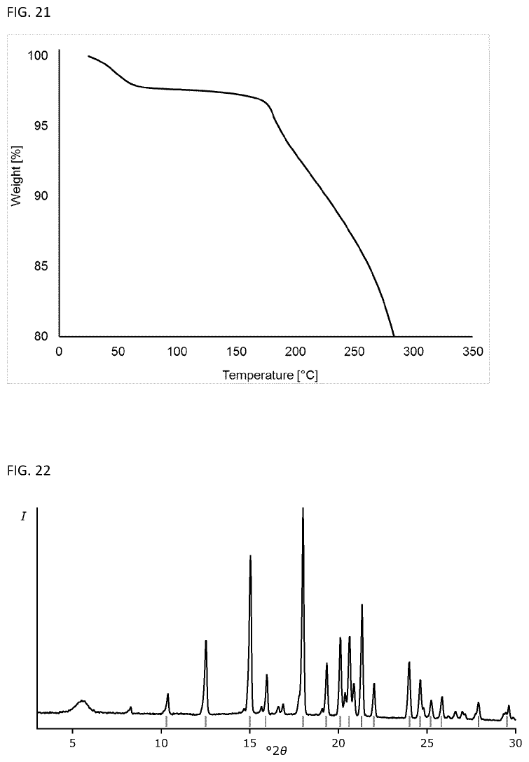

Other physical properties of boronic acid ester form NF5 include the following: Thermal behaviour of boronic acid ester form NF5 showed an exothermic decomposition peak in the DSC at temperatures greater than 140° C. accompanied by a weight loss step in the TGA. DSC and TGA profiles are displayed in FIGS. 20 and 21, respectively. DSC scan of boronic acid ester form NF5 was acquired on a Mettler-Toledo DSC 1 with a heating rate of 5 K/min, using nitrogen purge gas at 50 mL/min. TGA scan of boronic acid ester form NF5 was acquired on a Mettler-Toledo TGA 851 with a heating rate of 5 K/min, using nitrogen purge gas at 50 mL/min. Overall, boronic acid ester form NF5 revealed good solid-state properties (good crystallinity, high thermal stability).

In one embodiment, the invention provides for Compound 1 characterized as boronic acid ester form NF6.

In certain embodiments, boronic acid ester form NF6 is characterized by one or more 2θ peaks at 12.5, 15.0, 18.0, 20.6, and 21.3 degrees. In certain embodiments, boronic acid ester form NF6 is characterized by two or more 2θ peaks at 12.5, 15.0, 18.0, 20.6, and 21.3 degrees. In certain embodiments, boronic acid ester form NF6 is characterized by 2θ peaks at 12.5, 15.0, 18.0, 20.6, and 21.3 degrees.

In certain embodiments, boronic acid ester form NF6 is characterized by one or more 2θ peaks at 12.5, 15.0, 18.0, 19.3, 20.1, 20.6, 21.3 and 24.0 degrees. In certain embodiments, boronic acid ester form NF6 is characterized by two or more 2θ peaks at 12.5, 15.0, 18.0, 19.3, 20.1, 20.6, 21.3 and 24.0 degrees. In certain embodiments, boronic acid ester form NF6 is characterized by three or more 2θ peaks at 12.5, 15.0, 18.0, 19.3, 20.1, 20.6, 21.3 and 24.0 degrees. In certain embodiments, boronic acid ester form NF6 is characterized by four or more 2θ peaks at 12.5, 15.0, 18.0, 19.3, 20.1, 20.6, 21.3 and 24.0 degrees. In certain embodiments, boronic acid ester form NF6 is characterized by five or more 2θ peaks at 12.5, 15.0, 18.0, 19.3, 20.1, 20.6, 21.3 and 24.0 degrees. In certain embodiments, boronic acid ester form NF6 is characterized by six or more 2θ peaks at 12.5, 15.0, 18.0, 19.3, 20.1, 20.6, 21.3 and 24.0 degrees. In certain embodiments, boronic acid ester form NF6 is characterized by 2θ peaks at 12.5, 15.0, 18.0, 19.3, 20.1, 20.6, 21.3 and 24.0 degrees.

In certain embodiments, boronic acid ester form NF6 is characterized by 2θ peaks at

| | °2θ |
|---|---|
| | (Cu—Kα₁ |
| No. | radiation) ± 0.2° |
| 1 | 10.3 |
| 2 | 12.5 |
| 3 | 15.0 |
| 4 | 15.9 |
| 5 | 18.0 |
| 6 | 19.3 |
| 7 | 20.1 |
| 8 | 20.6 |
| 9 | 21.3 |
| 10 | 22.0 |
| 11 | 24.0 |
| 12 | 24.6 |
| 13 | 25.2 |
| 14 | 25.8 |
| 15 | 27.9 |
| 16 | 29.5 |

In another embodiment, boronic acid ester form NF6 is characterized by a diffraction pattern substantially similar to that of FIG. 22.

A Powder X-Ray Diffraction pattern of free base boronic acid ester form NF6 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In certain embodiments, boronic acid ester form NF6 is characterized by a crystal form, having a monoclinic space group P2₁ with the lattice parameters a=6.10505 Å, b=14.0122 Å, c=10.8234 Å, and α=γ=90°, β=99.137±0.1°. Reasonably rounded, the parameters are a=6.11±0.1 Å, b=14.01±0.1 Å, c=10.82±0.1 Å, and α=γ=90°±0.1°, β=99.14±0.1°. From the single crystal structure, it is clear that boronic acid ester form NF6 represents an anhydrous form of a boronic ester with methanol. Interestingly, the molecules show a ring closure (probably by coordination of electrons) between the boronic atom and the carbonyl oxygen. Single crystal X-Ray Structure data were obtained on boronic acid ester form NF6 as well (SuperNova diffractometer from Agilent, equipped with CCD Detector using Cu K_{α} radiation at 298 K).

In another embodiment, boronic acid ester form NF6 is characterized as a crystalline anhydrous form.

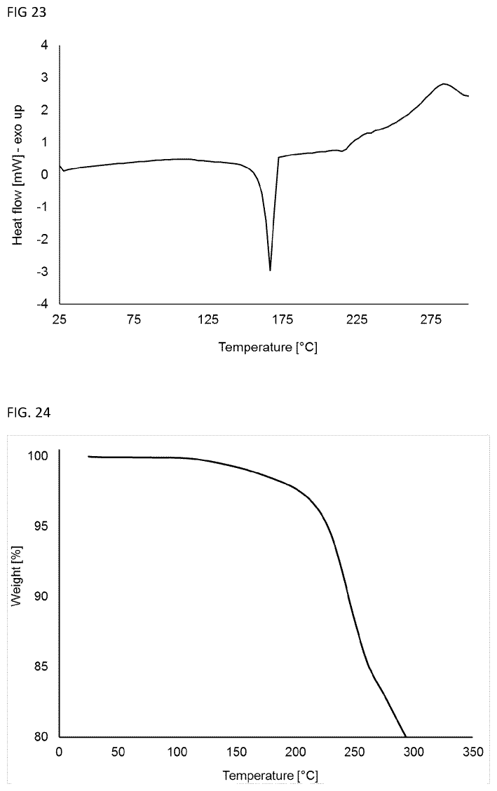

Other physical properties of boronic acid ester form NF6 include the following: Thermal behaviour of boronic acid ester form NF6 showed an endothermic melting/decomposition peak in the DSC at temperatures greater than 140° C. accompanied by weight loss events in the TGA. DSC and TGA profiles are displayed in FIGS. 23 and 24, respectively. DSC scan of boronic acid ester form NF6 was acquired on a Mettler-Toledo DSC 1 with a heating rate of 5 K/min, using nitrogen purge gas at 50 mL/min. TGA scan of boronic acid ester form NF6 was acquired on a Mettler-Toledo TGA 851 with a heating rate of 5 K/min, using nitrogen purge gas at 50 mL/min. Overall, boronic acid ester form NF6 revealed good solid-state properties (good crystallinity, high thermal stability).

In one embodiment, the invention provides for Compound 1 characterized as a hydrate of trimeric boronic acid adduct form NF7.

In certain embodiments, hydrate of trimeric boronic acid adduct form NF7 is characterized by one or more 2θ peaks at 10.5, 12.8, 17.2, 18.1, and 21.7 degrees. In certain embodiments, hydrate of trimeric boronic acid adduct form NF7 is characterized by two or more 2θ peaks at 10.5, 12.8, 17.2, 18.1, and 21.7 degrees. In certain embodiments, hydrate of trimeric boronic acid adduct form NF7 is characterized by 2θ peaks at 10.5, 12.8, 17.2, 18.1, and 21.7 degrees.

In certain embodiments, hydrate of trimeric boronic acid adduct form NF7 is characterized by one or more 2θ peaks at 10.5, 12.8, 17.2, 18.1, 19.2, 19.8 and 21.7 degrees. In certain embodiments, hydrate of trimeric boronic acid adduct form NF7 is characterized by two or more 2θ peaks at 10.5, 12.8, 17.2, 18.1, 19.2, 19.8 and 21.7 degrees. In certain embodiments, hydrate of trimeric boronic acid adduct form NF7 is characterized by three or more 2θ peaks at 10.5, 12.8, 17.2, 18.1, 19.2, 19.8 and 21.7 degrees. In certain embodiments, hydrate of trimeric boronic acid adduct form NF7 is characterized by four or more 2θ peaks at 10.5, 12.8, 17.2, 18.1, 19.2, 19.8 and 21.7 degrees. In certain embodiments, hydrate of trimeric boronic acid adduct form NF7 is characterized by five or more 2θ peaks at 10.5, 12.8, 17.2, 18.1, 19.2, 19.8 and 21.7 degrees. In certain embodiments, the hydrate of trimeric boronic acid adduct form NF7 is characterized by six or more 2θ peaks at 10.5, 12.8, 17.2, 18.1, 19.2, 19.8 and 21.7 degrees. In certain embodiments, hydrate of trimeric boronic acid adduct form NF7 is characterized by 2θ peaks at 10.5, 12.8, 17.2, 18.1, 19.2, 19.8 and 21.7 degrees.

In certain embodiments, the hydrate of trimeric boronic acid adduct form NF7 is characterized by 2θ peaks at

| | °2θ |
|---|---|
| | (Cu—Kα₁ |
| No. | radiation) ± 0.2° |
| 1 | 8.3 |
| 2 | 8.6 |
| 3 | 10.5 |
| 4 | 12.8 |
| 5 | 16.0 |
| 6 | 17.2 |
| 7 | 18.1 |
| 8 | 19.2 |
| 9 | 19.8 |
| 10 | 21.0 |
| 11 | 21.7 |
| 12 | 22.6 |
| 13 | 25.5 |

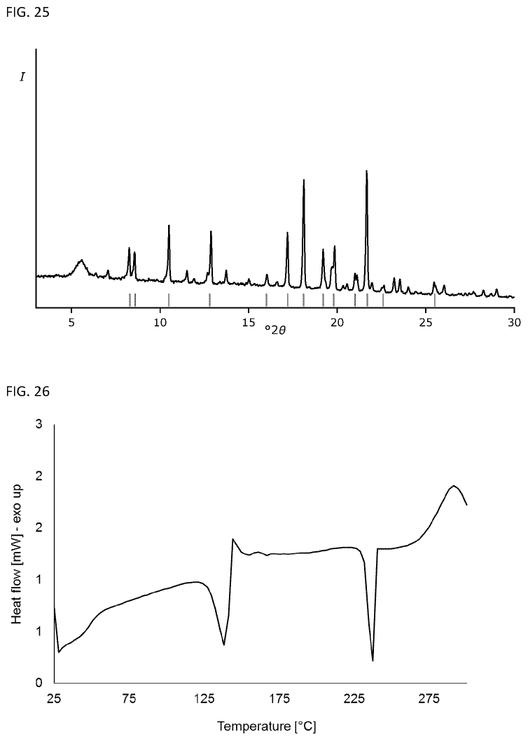

In another embodiment, the hydrate of trimeric boronic acid adduct form NF7 is characterized by a diffraction pattern substantially similar to that of FIG. 25.

A Powder X-Ray Diffraction pattern of the hydrate of trimeric boronic acid adduct form NF7 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In certain embodiments, the hydrate of trimeric boronic acid adduct form NF7 is characterized by a crystal form, having a monoclinic space group P2₁ with the lattice parameters a=14.015 Å, b=9.1829 Å, c=20.842 Å, and α=γ=90°, β=99.575°. Reasonably rounded, the parameters are a=14.02±0.1 Å, b=9.18±0.1 Å, c=20.84±0.1 Å, and α=γ=90°±0.1°, β=99.58±0.1°. From the single crystal structure, it is clear form NF7 represents a hydrate form of a trimeric boronic acid adduct. Interestingly, one of the three molecular units shows a bond to a pyridine molecule. Single crystal X-Ray Structure data were obtained on the hydrate of trimeric boronic acid adduct form NF7 as well (SuperNova diffractometer from Agilent, equipped with CCD Detector using Cu K_{α} radiation at 298 K)

In another embodiment, the hydrate of trimeric boronic acid adduct form NF7 is characterized as a crystalline mono-hydrate form.

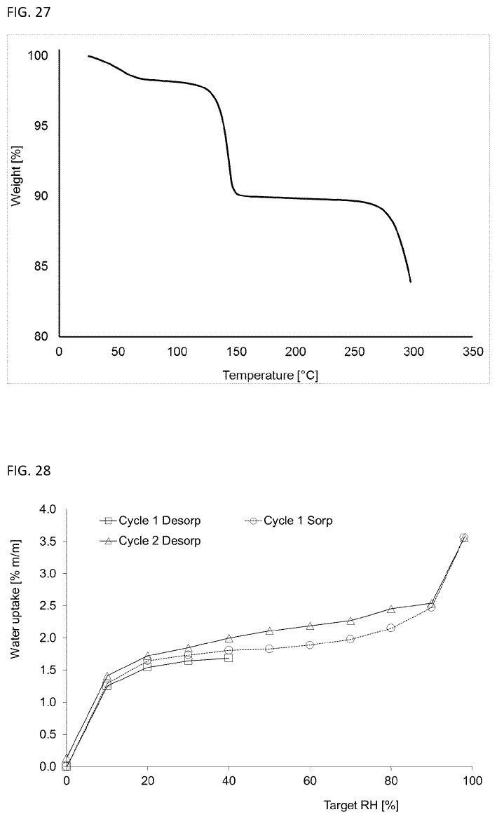

Other physical properties of the hydrate of trimeric boronic acid adduct form NF7 include the following: Thermal behaviour of the hydrate of trimeric boronic acid adduct form NF7 showed an endothermic peak in the DSC at temperatures greater than 100° C., accompanied by a weight loss step in the TGA. This is followed by an exothermic event in the DSC, assigned to a phase transition to trimeric form A1. Finally, the melting of trimeric form A1 with melting peak onset at temperatures greater than 220° C. DSC and TGA profiles are displayed in FIGS. 26 and 27, respectively. DSC scan of the hydrate of trimeric boronic acid adduct form NF7 was acquired on a Mettler-Toledo DSC 1 with a heating rate of 5 K/min, using nitrogen purge gas at 50 mL/min. TGA scan of the hydrate of trimeric boronic acid adduct form NF7 was acquired on a Mettler-Toledo TGA 851 with a heating rate of 5 K/min, using nitrogen purge gas at 50 mL/min. Water Vapour Sorption behaviour of the hydrate of trimeric boronic acid adduct form NF7 revealed small water uptake levels≤1% m/m in the relative humidity (rh) range 0-80% rh, and slightly elevated water uptake levels≤5% m/m in the relative humidity (rh) range 90-98% rh. The hydrate of trimeric boronic acid adduct form NF7 can be classified as slightly hygroscopic acc. to Ph. Eur. Criteria (section 5.11.). Water Vapor Sorption isotherm (25° C.) of the hydrate of trimeric boronic acid adduct form NF7 is displayed in FIG. 28. Water Vapour Sorption isotherm was acquired on a DVS-Intrinsic system from SMS. Overall, the hydrate of trimeric boronic acid adduct form NF7 revealed good solid-state properties (good crystallinity, slightly hygroscopic, high thermal stability).

In one embodiment, the invention provides for Compound 1 characterized as boronic acid ester form NF8.

In certain embodiments, boronic acid ester form NF8 is characterized by one or more 2θ peaks at 9.4, 10.3, 15.8 and 17.5 degrees. In certain embodiments, boronic acid ester form NF8 is characterized by two or more 2θ peaks at 9.4, 10.3, 15.8 and 17.5 degrees. In certain embodiments, boronic acid ester form NF8 is characterized by 2θ peaks at 9.4, 10.3, 15.8 and 17.5 degrees.

In certain embodiments, boronic acid ester form NF8 is characterized by one or more 2θ peaks at 9.4, 10.3, 12.8, 14.8, 15.8, 17.5, 21.6 and 22.1 degrees. In certain embodiments, boronic acid ester form NF8 is characterized by two or more 2θ peaks at 9.4, 10.3, 12.8, 14.8, 15.8, 17.5, 21.6 and 22.1 degrees. In certain embodiments, boronic acid ester form NF8 is characterized by three or more 2θ peaks at 9.4, 10.3, 12.8, 14.8, 15.8, 17.5, 21.6 and 22.1 degrees. In certain embodiments, boronic acid ester form NF8 is characterized by four or more 2θ peaks at 9.4, 10.3, 12.8, 14.8, 15.8, 17.5, 21.6 and 22.1 degrees. In certain embodiments, boronic acid ester form NF8 is characterized by five or more 2θ peaks at 9.4, 10.3, 12.8, 14.8, 15.8, 17.5, 21.6 and 22.1 degrees. In certain embodiments, boronic acid ester form NF8 is characterized by six or more 2θ peaks at 9.4, 10.3, 12.8, 14.8, 15.8, 17.5, 21.6 and 22.1 degrees. In certain embodiments, boronic acid ester form NF8 is characterized by seven or more 2θ peaks at 9.4, 10.3, 12.8, 14.8, 15.8, 17.5, 21.6 and 22.1 degrees. In certain embodiments, boronic acid ester form NF8 is characterized by 2θ peaks at 9.4, 10.3, 12.8, 14.8, 15.8, 17.5, 21.6 and 22.1 degrees.

In certain embodiments, boronic acid ester form NF8 is characterized by 2θ peaks at

| | °2θ |
|---|---|
| | (Cu—Kα₁ |
| No. | radiation) ± 0.2° |
| 1 | 6.3 |
| 2 | 9.4 |
| 3 | 10.3 |
| 4 | 12.8 |
| 5 | 14.8 |
| 6 | 15.8 |
| 7 | 16.8 |
| 8 | 17.5 |
| 9 | 19.4 |
| 10 | 19.7 |
| 11 | 20.0 |
| 12 | 20.7 |
| 13 | 21.6 |
| 14 | 22.1 |
| 15 | 22.6 |
| 16 | 23.1 |
| 17 | 23.6 |
| 18 | 24.2 |
| 19 | 27.8 |
| 20 | 29.1 |

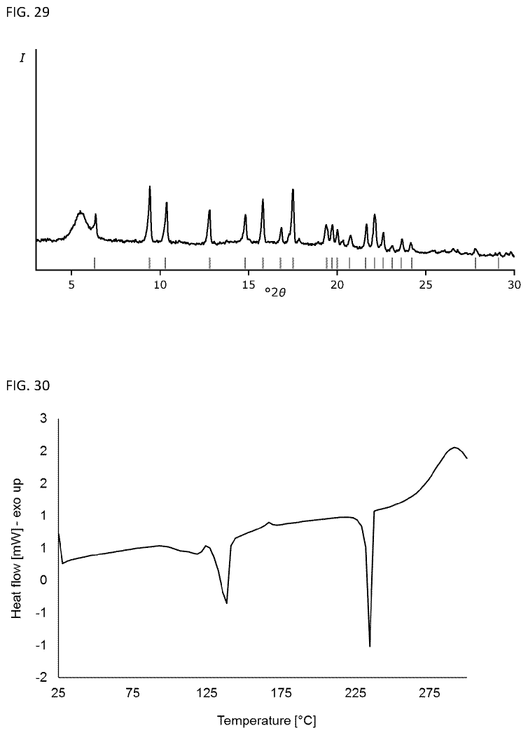

In another embodiment, boronic acid ester form NF8 is characterized by a diffraction pattern substantially similar to that of FIG. 29.

A Powder X-Ray Diffraction pattern of free base boronic acid ester form NF8 was obtained by standard techniques as described in the European Pharmacopeia 6^{th} Edition chapter 2.9.33, and was characterised by the following X-ray powder diffractogram (monochromatic Cu-Kα₁ radiation, λ=1.5406 Å, Stoe StadiP 611 KL transmission diffractometer).

In another embodiment, boronic acid ester form NF8 is characterized as a crystalline anhydrous form.

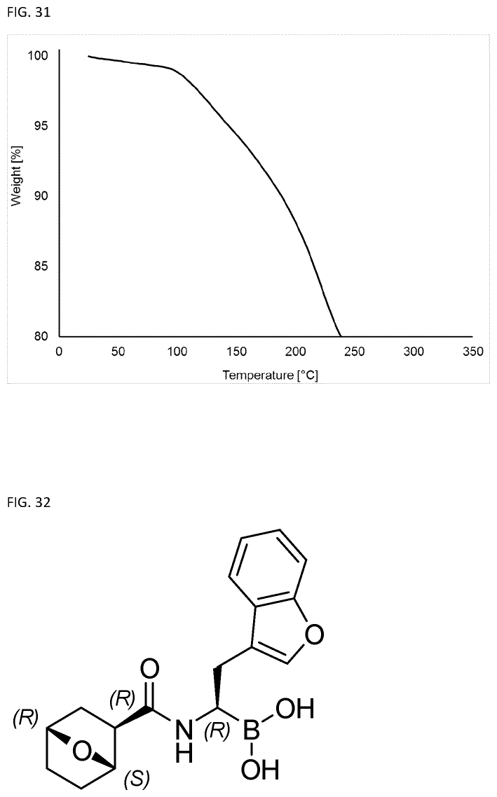

Other physical properties of boronic acid ester form NF8 include the following: Thermal behaviour of boronic acid ester form NF8 showed an endothermic peak in the DSC at temperatures over 100° C., accompanied by a weight loss step in the TGA. This is followed by an exothermic event in the DSC, assigned to a phase transition to the anhydrous trimeric adduct form A1. Finally, melting of trimeric form A1 occurs at temperatures above 220° C. DSC and TGA profiles are displayed in FIGS. 30 and 31, respectively. DSC scan of boronic acid ester form NF8 was acquired on a Mettler-Toledo DSC 1 with a heating rate of 5 K/min, using nitrogen purge gas at 50 mL/min. TGA scan of boronic acid ester form NF8 was acquired on a Mettler-Toledo TGA 851 with a heating rate of 5 K/min, using nitrogen purge gas at 50 mL/min. Overall, boronic acid ester form NF8 revealed good solid-state properties (good crystallinity, high thermal stability).

In one embodiment, the invention provides for Compound 1 characterized as a mixture of crystalline forms A1 and A2.

The development of solid-state preparation routes was mainly based on solvent crystallisation approaches to enable scalability to large scale as well as providing powder material with good manufacturability properties.

A mixture of morphic forms is not favorable from a regulatory and quality perspective, as phase compositions of mixtures are challenging to control from batch to batch. Variability of phase compositions requires extensive characterisation to assess impact on critical quality attributes (e.g. oral absorption behavior, stability behavior) and may also jeopardise robust DP manufacturability if parameters such as particle habit are different for different forms and mixtures thereof.

Surprisingly, the invention provides preparation routes for the thermodynamically stable phase-pure crystalline form A1 of Compound 1, which provides powder material with good manufacturability properties in large scale.

In another aspect, the invention features a pharmaceutical composition comprising any of the forms described above, and a pharmaceutically acceptable carrier. In another embodiment, the pharmaceutical composition further comprises an additional therapeutic agent.

In another aspect, the invention features a process of preparing Form A1 comprising dissolving Compound 1 in an organic solvent or water. In one aspect of this embodiment, the invention features a process of preparing form A1 comprising dissolving Compound 1 in an organic solvent or a mixture of organic solvents. In certain embodiments, the organic solvent is methanol, Ethanol, 1-Propanol, 2-Propanol, 2-Butanol, dichloromethane, ethyl acetate, Acetone, DMSO, DMA, Methyl Isobutyl Ketone, Methyl Acetate, 1,4-Dioxane, Di ethyl ether, Methyl tert butyl ether, Tetrahydrofuran, Acetonitrile, Dichloromethane, Chloroform, Pyridine, or Toluene or a mixture of organic solvents. In certain embodiments, the solvent is Methanol, 1-Propanol, 2-Propanol, Pyridine, 1-Butanol, Iso-Butanol, Acetone, Ethyl acetate, or Dichloromethane, or a mixture thereof.

In certain embodiments, the solvent is Dichloromethane or Ethyl acetate, or a mixture thereof.

In certain embodiments, Compound 1 is dissolved in the organic solvent between about 20 and 75° C. In certain embodiments, Compound 1 is dissolved in the organic solvent at about 25° C. In certain embodiments, Compound 1 is dissolved in the organic solvent at about 50° C.

In certain embodiments, the invention features a process of preparing Form A1 comprising dissolving Compound 1 in dichloromethane, acetone, ethyl acetate or mixtures thereof.

In certain embodiments, the process comprises suspending Compound 1 in water until spontaneous crystallization occurs.

In certain embodiments, the process comprises dissolving Compound 1 in an organic solvent and water mixture. In one aspect of this embodiment, the organic solvent is selected from acetonitrile, dimethyl sulfoxide (DMSO), or dimethylacetamide (DMA). In some aspects of this embodiment, the organic solvent to water ratio is about 1:1.

In certain embodiments, the process comprises crystallization of Compound 1 from an alcohol. In certain embodiments, the alcohol is methanol, n-butanol, iso-butanol, or 2-propanol.

In certain embodiments, the process comprises dissolving Compound 1 in a binary mixture with pyridine. In one aspect of this embodiment, the pyridine is mixed with water. In another aspect of this embodiment, the pyridine is mixed with methyl acetate. In a third aspect, the pyridine is mixed with ethyl acetate. In a fourth aspect, the pyridine is mixed with methyl isobutyl ketone.

In certain embodiments, the compounds and solid forms of the invention were synthesized in accordance with the schemes provided in the Examples below.

### 4. Uses, Formulation and Administration

### Pharmaceutically Acceptable Compositions

According to another embodiment, the invention provides a composition comprising a solid form of compound 1 of this invention or a pharmaceutically acceptable derivative thereof and a pharmaceutically acceptable carrier, adjuvant, or vehicle. The amount of solid form of compound 1 in compositions of this invention is such that is effective to measurably inhibit LMP7, in a biological sample or in a patient. In certain embodiments, a composition of this invention is formulated for administration to a patient in need of such composition.

The term “patient” or “subject”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the solid form of the compound with which it is formulated. Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

In certain embodiments, the invention provides a method for inhibiting LMP7 in a patient in need thereof comprising the step of administering to said patient a solid form of compound 1 according to the invention.

In certain embodiments, the invention is directed to the use of a solid form of compound 1 for modulating or inhibiting a LMP7 enzyme. The term “modulation” denotes any change in LMP7-mediated signal transduction, which is based on the action of the specific inventive compounds capable to interact with the LMP7 target in such a manner that makes recognition, binding and activating possible. The compounds are characterized by such a high affinity to LMP7, which ensures a reliable binding of LMP7. In certain embodiments, the substances are highly selective for LMP7 over most other kinases in order to guarantee an exclusive and directed recognition with the single LMP7 target. In the context of the present invention, the term “recognition”—without being limited thereto—relates to any type of interaction between the specific compounds and the target, particularly covalent or non-covalent binding or association, such as a covalent bond, hydrophobic/hydrophilic interactions, van der Waals forces, ion pairs, hydrogen bonds, ligand-receptor (enzyme-inhibitor) interactions, and the like. Such association may also encompass the presence of other molecules such as peptides, proteins or nucleotide sequences. The present protein/ligand (enzyme-inhibitor)-interaction is characterized by high affinity, high selectivity and minimal or even lacking cross-reactivity to other target molecules to exclude unhealthy and harmful impacts to the treated subject.

In certain embodiments, the present invention relates to a method for inhibiting a LMP7 enzyme, with at least a solid form of compound 1 under conditions such that said LMP7 enzyme is inhibited. In certain embodiments, the system is a cellular system. In other embodiments, the system is an in-vitro translation which is based on the protein synthesis without living cells. The cellular system is defined to be any subject provided that the subject comprises cells. Hence, the cellular system can be selected from the group of single cells, cell cultures, tissues, organs and animals. In certain embodiments, the method for modulating a LMP7 enzyme is performed in-vitro. The prior teaching of the present specification concerning a solid form of compound 1 including any embodiments thereof, is valid and applicable without restrictions to the compounds when used in the method for inhibiting LMP7. The prior teaching of the present specification concerning a solid form of compound 1 is valid and applicable without restrictions to the compounds when used in the method for inhibiting LMP7.

Provided solid forms of compound 1 are inhibitors of LMP7 and are therefore useful for treating one or more disorders associated with activity of LMP7. Thus, in some embodiments, the present invention provides a method for treating a LMP7-mediated disorder comprising the step of administering to a patient in need thereof a solid form of compound 1.

As used herein, the term “LMP7-mediated” disorders or conditions as used herein means any disease or other deleterious condition in which LMP7 is known to play a role. Accordingly, another embodiment of the present invention relates to treating or lessening the severity of one or more diseases in which LMP7 is known to play a role. Specifically, the present invention relates to a method of treating or lessening the severity of a disease or condition selected from a proliferative disorder or an autoimmune disorder, wherein said method comprises administering to a patient in need thereof a compound or composition according to the present invention.

In some embodiments, the present invention provides a method for treating or lessening the severity of one or more diseases and conditions associated with LMP7, wherein the disease or condition is selected from a cancer. In one embodiment, the cancer is a B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis. In some embodiments, the cancer is breast cancer, prostate cancer, or cancer of the mast cells (e.g., mastocytoma, mast cell leukemia, mast cell sarcoma, systemic mastocytosis). In one embodiment, the cancer is bone cancer. In another embodiment, the cancer is of other primary origin and metastasizes to the bone. In certain embodiments, the cancer is colorectal cancer or pancreatic cancer. In certain embodiments, the cancer is cancer is melanoma, glioma, glioblastomas, or cancer of the breast, lung, bladder, esophagus, stomach, colon, head, neck, ovary, prostate, pancreas, rectum, endometrium, or liver. In other embodiments, the cancer is triple-negative breast cancer, non-small cell lung cancer, and head and neck carcinoma.

In certain embodiments, the cancer is multiple myeloma. In one aspect of this embodiment, the subject with multiple myeloma has a t(4; 14) and/or t(14; 16) translocation.

In certain embodiments, the cancer is hematological malignancy selected from mantle cell lymphoma (MCL), T cell leukemia/lymphoma, acute myeloid leukemia (AML), acute lymphoblastic leukemia (ALL), diffuse large B-cell lymphoma (DLBCL), chronic lymphocytic leukemia (CLL), chronic myelogenous leukemia (CML), follicular lymphoma (FL) or marginal zone B-cell lymphoma (MZL).

In certain embodiments, the LMP7-mediated disorder is monoclonal gammopathy of uncertain significance (MGUS); smoldering multiple myeloma (SMM); and/or solitary plasmacytoma.

In other embodiments, the LMP7-mediated disorder is plasmacyotma, lymphoplasmacytic lymphoma, amyloid light-chain (AL) amyloidosis, and/or Waldenstrom's macroglobulinemia (WM).

In some embodiments, the present invention provides a method for treating or lessening the severity of one or more diseases and conditions associated with LMP7. In some embodiments, the disease or condition is an autoimmune disease, e.g., systemic lupus erythematosis, chronic rheumatoid arthritis, inflammatory bowel disease, multiple sclerosis, amyotrophic lateral sclerosis (ALS), atherosclerosis, scleroderma, autoimmune hepatitis, Sjogren Syndrome, lupus nephritis, glomerulonephritis, Rheumatoid Arthritis, Psoriasis, Myasthenia Gravis, Imunoglobuline A nephropathy, Vasculitis, Transplant rejection, Myositis, Henoch-Schönlein purpura and asthma; and wherein the hematological malignancy is a disease selected from the group consisting of: multiple myeloma, mantle cell lymphoma, diffuse large B-cell lymphoma, plasmocytoma, follicular lymphoma, immunocytoma, acute lymphoblastic leukemia, chronic lymphocytic leukemia and myeloid leukemia; and wherein the solid tumor is selected form a group consisting of: inflammatory breast and colon cancer, lung cancer, head and neck cancer, prostate cancer, pancreas cancer, bladder cancer, renal cancer, hepatocellular cancer and gastric cancer. In certain embodiments, the disease or condition is systemic lupus erythematosus (SLE or lupus) or lupus nephritis.

In some embodiments, the present invention provides a method for treating or lessening the severity of one or more diseases and conditions associated with LMP7, wherein the disease or condition is selected from an autoimmune or chronic inflammatory disease selected from the group consisting of: systemic lupus erythematosis, chronic rheumatoid arthritis, inflammatory bowel disease, multiple sclerosis, amyotrophic lateral sclerosis (ALS), atherosclerosis, scleroderma, autoimmune hepatitis, Sjogren Syndrome, lupus nephritis, glomerulonephritis, Rheumatoid Arthritis, Psoriasis, Myasthenia Gravis, Imunoglobuline A nephropathy, Vasculitis, Transplant rejection, Myositis, Henoch-Schönlein Purpura and asthma; cancer is preferably a hematological malignancy or a solid tumor, wherein the hematological malignancy is preferably a disease selected from the group of malignant B- and T/NK-cell non-Hodgkin lymphoma such as: multiple myeloma, mantle cell lymphoma, diffuse large B-cell lymphoma, plasmocytoma, follicular lymphoma, immunocytoma, acute lymphoblastic leukemia, chronic lymphocytic leukemia and myeloid leukemia; and wherein the solid tumor is preferably a disease selected from the group of: inflammatory breast, liver and colon cancer, lung cancer, head and neck cancer, prostate cancer, pancreas cancer, bladder cancer, renal cancer, hepatocellular cancer and gastric cancer.

In some embodiments, the present invention provides a method for treating or lessening the severity of one or more diseases and conditions associated with LMP7, wherein the disease or condition is selected from heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

It is another object of the invention to provide a method for treating diseases that are caused, mediated and/or propagated by LMP7 activity, wherein a solid form of compound 1 is administered to a mammal in need of such treatment. In certain embodiments, the invention provides a method for treating lupus, wherein a solid form of compound 1 is administered to a mammal in need of such treatment. In certain embodiments, the compound is administered in an effective amount as defined above. In certain embodiments, the treatment is an oral administration.

The method of the invention can be performed either in-vitro or in-vivo. The susceptibility of a particular cell to treatment with the compounds according to the invention can be particularly determined by in-vitro tests, whether in the course of research or clinical application. Typically, a culture of the cell is combined with a compound according to the invention at various concentrations for a period of time which is sufficient to allow the active agents to inhibit LMP7 activity, usually between about one hour and one week. In-vitro treatment can be carried out using cultivated cells from a biopsy sample or cell line.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

For identification of a signal transduction pathway and for detection of interactions between various signal transduction pathways, various scientists have developed suitable models or model systems, for example cell culture models and models of transgenic animals. For the determination of certain stages in the signal transduction cascade, interacting compounds can be utilized in order to modulate the signal. The compounds according to the invention can also be used as reagents for testing LMP7-dependent signal transduction pathways in animals and/or cell culture models or in the clinical diseases mentioned in this application.

Moreover, the subsequent teaching of the present specification concerning the use of a solid form of compound 1 for the production of a medicament for the prophylactic or therapeutic treatment and/or monitoring is considered as valid and applicable without restrictions to the use of the compound for the inhibition of LMP7 activity if expedient.

The invention also relates to the use of a solid form of compound 1, or pharmaceutically acceptable salts thereof for the therapeutic treatment and/or monitoring of diseases that are caused, mediated and/or propagated by LMP7 activity. Furthermore, the invention relates to the use of a solid form of compound 1 for the production of a medicament for the therapeutic treatment and/or monitoring of diseases that are caused, mediated and/or propagated by LMP7 activity. In certain embodiments, the invention provides the use of a solid form of compound 1 for the production of a medicament for the therapeutic treatment of a LMP7-mediated disorder.

The solid form of compound 1 can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease completely. Either the identical compound or different compounds can be applied. The methods of the invention can also be used to reduce the likelihood of developing a disorder or even prevent the initiation of disorders associated with LMP7 activity in advance or to treat the arising and continuing symptoms.

The invention furthermore relates to a medicament comprising at least one solid form of compound 1.

A “medicament” in the meaning of the invention is any agent in the field of medicine, which comprises Compound 1 or preparations thereof (e.g. a pharmaceutical composition or pharmaceutical formulation) and can be used in prophylaxis, therapy, follow-up or aftercare of patients who suffer from diseases, which are associated with LMP7 activity, in such a way that a pathogenic modification of their overall condition or of the condition of particular regions of the organism could establish at least temporarily.

In various embodiments, the active ingredient may be administered alone or in combination with other treatments. A synergistic effect may be achieved by using more than one compound in the pharmaceutical composition, i.e. the compound of formula (I) is combined with at least another agent as active ingredient, which is either another compound of formula (I) or a compound of different structural scaffold. The active ingredients can be used either simultaneously or sequentially.

Included herein are methods of treatment in which at least one chemical entity provided herein is administered in combination with one or more therapeutic agent. In one aspect of this embodiment, the one or more additional therapeutic agents is an EGFR pathway inhibitor, MAPK pathway inhibitor, XPO1 inhibitor, a DNA repair pathway inhibitor, FGFR pathway inhibitor, PI3K/AKT/mTOR pathway inhibitor, and/or MCL1 inhibitor.

Examples of the EGFR pathway inhibitor is selected from Erlotinib, Afatinib, Gefitinib, Cetuximab, Panitumumab, Lapatinib, Osimertinib, Trastuzumab, and/or Pertuzumab.

In some embodiments, the anti-inflammatory agent is a salicylate. Salicylates include by are not limited to acetylsalicylic acid or aspirin, sodium salicylate, and choline and magnesium salicylates.

Examples of the MAPK pathway inhibitor is selected from Trametinib, Cobimetinib, Binimetinib, Selumetinib, Refametinib, Pimasertib, AMG 510, MRTX849, Vemurafenib, Dabrafenib, Encorafenib, LXH254, HM95573, XL281, RAF265, RAF709, LY3009120, Ulixertinib, SCH772984, TNO155, RMC-4630, JAB-3068, JAB-3312, AMG-510, MRTX849, LY3499446 and/or BI 1701963.

Examples of the XPO1 inhibitor is selected from Selinexor and/or KPT-8602.

Examples of the DNA repair pathway inhibitor is selected from M3541, M4076, BAY1895344, NOV1401, E7016, BGB-290, CEP-9722, Olaparib, Rucaparib, Niraparib, and/or Talazoparib.

Examples of the FGFR pathway inhibitor is selected from Erdafitinib, AZD4547, LY2874455, Debio 1347, NVP-BGJ398, Pemigatinib, Rogaratinib, PRN1371, TAS-120, and/or Nintedanib.

Examples of the PI3K/AKT/mTOR pathway inhibitor is selected from Rapamycin, Temsirolimus, Everolimus, Ridaforolimus, Alpelisib, Idelalisib, Copanlisib, Duvelisib, MK-2206, and/or AZD5363.

Examples of the MCL1 inhibitor is selected from A-1210477, VU661013, AZD5991, AMG-176, AMG-397, S63845, S64315, Venetoclax, HDM201, NVP-CGM097, RG-7112, MK-8242, RG-7388, SAR405838, AMG-232, DS-3032, RG7775, and/or APG-115.

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined above may be applied as a monotherapy or may involve, in addition to the herein disclosed compounds of formula I, conventional surgery or radiotherapy or medicinal therapy. Such medicinal therapy, e.g. a chemotherapy or a targeted therapy, may include one or more, but preferably one, of the following anti-tumor agents:
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine^{1,3};
Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur^{2,3}, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide^{1,3};
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴;
Photosensitizers: such as methoxsalen³; porfimer sodium, talaporfin, temoporfin; Antibodies: such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3}; catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};
Cytokines: such as aldesleukin, interferon alfa², interferon alfa2a³, interferon alfa2b^{2,3}; celmoleukin, tasonermin, teceleukin, oprelvekin^{1,3}, recombinant interferon beta-1a⁴;
Drug Conjugates: such as denileukin diftitox, ibritumomab tiuxetan, iobenguane I123, prednimustine, trastuzumab emtansine, estramustine, gemtuzumab, ozogamicin, aflibercept; cintredekin besudotox, edotreotide, inotuzumab ozogamicin, naptumomab estafenatox, oportuzumab monatox, technetium (99mTc) arcitumomab^{1,3}, vintafolide^{1,3};
Vaccines: such as sipuleucel³; vitespen³, emepepimut-S³, oncoVAX⁴, rindopepimut³, troVax⁴, MGN-1601⁴, MGN-1703⁴; and
Miscellaneous: alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴. (¹ Prop. INN (Proposed International Nonproprietary Name); ² Rec. INN (Recommended International Nonproprietary Names); ³ USAN (United States Adopted Name); ⁴ no INN).

In another aspect, the invention provides for a kit consisting of separate packs of an effective amount of a compound according to the invention and an effective amount of a further active ingredient. The kit comprises suitable containers, such as boxes, individual bottles, bags or ampoules. The kit may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further active ingredient in dissolved or lyophilized form.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The compounds of the invention are characterized by a high specificity and stability, low manufacturing costs and convenient handling. These features form the basis for a reproducible action, wherein the lack of cross-reactivity is included, and for a reliable and safe interaction with the target structure.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

The symbols and conventions used in the following descriptions of processes, schemes, and examples are consistent with those used in the contemporary scientific literature, for example, the Journal of the American Chemical Society or the Journal of Biological Chemistry.

All forms were characterized according to standard methods which are found in e.g. Rolf Hilfiker, ‘Polymorphism in the Pharmaceutical Industry’, Wiley-VCH. Weinheim 2006 (Chapter 6: X-Ray Diffraction, Chapter 6: Vibrational Spectroscopy, Chapter 3: Thermal Analysis, Chapter 9: Water Vapour Sorption, and references therein); and H. G. Brittain, ‘Polymorphism in Pharmaceutical Solids, Vol. 95, Marcel Dekker Inc., New York 1999 (Chapter 6 and references therein).

Unless otherwise indicated, all temperatures are expressed in ° C. (degrees Centigrade). All reactions were conducted at room temperature unless otherwise noted. All compounds of the present invention were synthesized by processes developed by the inventors.

¹H-NMR spectra were recorded on a Bruker Avance III 400 MHz. Chemical shifts are expressed in parts per million (ppm, δ units). Coupling constants are in units of Hertz (Hz). Splitting patterns describe apparent multiplicities and are designated as s (singlet), d (doublet), t (triplet), q (quartet), m (multiplet), or br (broad).

Mass spectra were obtained on Agilent 1200 Series mass spectrometers from Agilent technologies, using either Atmospheric Chemical Ionization (APCI) or Electrospray Ionization (ESI). Column: XBridge C8, 3.5 μm, 4.6×50 mm; Solvent A: water+0.1% TFA; Solvent B: CAN; Flow: 2 ml/min; Gradient: 0 min: 5% B, 8 min: 100% B, 8.1 min: 100% B, 8.5 min: 5% B, 10 min 5% B.

HPLC data were obtained using Agilent 1100 series HPLC from Agilent technologies using XBridge column (C8, 3.5 μm, 4.6×50 mm). Solvent A: water+0.1% TFA; Solvent B: ACN; Flow: 2 ml/min; Gradient: 0 min: 5% B, 8 min: 100% B, 8.1 min: 100% B, 8.5 min: 5% B, 10 min 5% B.

The microwave reactions were conducted using Biotage Initiator Microwave Synthesizer using standard protocols that are known in the art.

Some abbreviations that may appear in this application are as follows:

| | |
|---|---|
| δ | chemical shift |
| API | Active pharmaceutical ingredient |
| d | deuterium or doublet |
| dd | doublet of doublets |
| DCM | dichloromethane |
| DMF | dimethylformamide |
| DMSO | dimethylsulfoxide |
| THF | tetrhydrofuran |
| eq. | equivalent |
| h | hour |
| ¹H | proton |
| HPLC | high pressure liquid chromatography |
| J | coupling constant |
| LC | liquid chromatography |
| m | multiplet |
| M | molecular ion |
| MHz | Megahertz |
| min | minute |
| mL | milliliter |
| MS | mass spectrometry |
| m/z | mass-to-charge ratio |
| NMR | nuclear magnetic resonance |
| RBF | Round Bottom Flask |
| RT | room temperature |
| s | singlet |
| TLC | thin layer chromatography |
| UV | ultraviolet |

Compound numbers utilized in the Examples below correspond to compound numbers set forth supra.

### Example 1: Formation of Anhydrous Trimeric Adduct Form A1

a) Small Scale: Precipitation from Ethyl Acetate

Approximately 2.2 g oily substance, obtained from Dichloromethane phase after extraction were diluted with 100 mL Ethyl acetate. The solution was treated by ultrasound for approximately one minute and the obtained precipitate was filtered and dried at 60° C. under vacuum.

¹H NMR (500 MHz, DMSO-d6//D20) 7.60 (s, 1H), 7.60-7.56 (m, 1H), 7.47 (d, J=8.1 Hz, 1H), 7.27 (td, J=8.3, 7.8, 1.3 Hz, 1H), 7.21 (td, J=7.5, 0.9 Hz, 1H), 4.47 (t, J=4.7 Hz, 1H), 4.41 (d, J=4.5 Hz, 1H), 3.08 (dd, J=8.2, 6.0 Hz, 1H), 2.84 (dd, J=14.8, 5.8 Hz, 1H), 2.72 (dd, J=15.0, 8.3 Hz, 1H), 2.46 (dd, J=9.0, 4.9 Hz, 1H), 1.76-1.69 (m, 1H), 1.61 (dd, J=11.9, 9.1 Hz, 1H), 1.53-1.34 (m, 4H)b)

### Small Scale: Evaporation Crystallization

Approximately 14-15 mg of the substance were dissolved in 3 mL Acetone or Ethyl acetate at 50° C. The solution was filtered using a 0.2 μm PTFE syringe filter. After that, from the obtained clear solution the solvent was evaporated at 50° C. to dryness.

### c) Gram Scale: Combination of Anti-Solvent and Evaporation Crystallization

Approximately 5.0 g of the substance were weighed in a 100 mL reactor of the synthesis workstation EasyMax 402™ from Mettler Toledo. The substance was dissolved in 60 mL Dichloromethane at a reactor temperature of 25° C. by stirring (propeller) at 200 rpm. With a dosing unit 30 mL ethyl acetate was added to the clear solution at 1 mUmin. After addition of 41 mg seed (form A1, suspended in 1 mL ethyl acetate) the solution became turbid. Further 20 mL of ethyl acetate were pumped to the reactor at 1 mU/min. Then, 45 mL of Dichloromethane were evaporated at 0.75 mUmin using a dry nitrogen flow. After evaporation was finished, 25 mL ethyl acetate were added to the obtained suspension at 1 mUmin. The suspension was further stirred for 30 min. Solid liquid separation was done by vacuum filtration using a Buchner funnel and cellulose filter paper (Whatman grade 2). The filter cake was washed with 10 mL ethyl acetate and the final crystallisate was dried at 50° C. using a dry nitrogen flow for 24 hours.

¹H NMR (500 MHz, DMSO-d6/D20) d 7.62 (s, 1H), 7.63-7.58 (m, 2H), 7.48 (d, J=8.1 Hz, 1H), 7.27 (dd, J=7.3, 1.3 Hz, 1H), 7.22 (td, J=7.5, 0.9 Hz, 1H), 4.46 (t, J=4.7 Hz, 1H), 4.42 (d, J=4.5 Hz, 1H), 3.15 (dd, J=8.1, 6.0 Hz, 1H), 2.86 (dd, J=14.8, 5.9 Hz, 1H), 2.75 (dd, J=14.9, 8.2 Hz, 1H), 2.44 (dd, J=9.0, 4.9 Hz, 1H), 1.80-1.73 (m, 1H), 1.58 (dd, J=11.9, 9.1 Hz, 1H), 1.53-1.34 (m, 4H)

It should be noted that due to the presence of water in the DMSO in the 1HNMR the monomer is reformed. So this is essentially the 1HNMR of the monomer of cpd 1.

### Large Scale: GMP Batch

Respective amount of drug substance, directly obtained from last synthesis step as dichloromethane solution (12.5 volume), was charged into the reactor. The organic solvent was distilled under vacuum at external temperature 35±5° C. to obtain an oil. In process control (NMR) was carried out during evaporation step until content of dichloromethane was ≤20%. 2.5 volumes of dichloromethane were added to the obtained oil and stirred at external temperature 25±5° C. until a clear solution was achieved. 3.5 volumes ethyl acetate were added to the solution and stirred at external temperature 25±5° C. Then the solution was heated up by setting the external temperature to 55±5° C. After spontaneous precipitation, the suspension was stirred for at least 1 hour at the same temperature. Residual dichlormethane was evaporated under vacuum, then further ethyl acetate was added (1.0 volume). Suspension was cooled down at linear temperature ramp from 55±5° C. (external) to 0±5° C. (external) within 8 hours. At external temperature 0±5° C. the suspension was stirred for 0.5 hours. Solid/liquid separation was done by filtration and filter cake was washed with ethyl acetate (1.0 volume). Obtained solid material was dried in an oven under vacuum at external temperature 60±5° C. for 12 hours. 3.5 kilogram of form A1 were obtained.

¹H NMR (500 MHz, DMSO-d6/D20) 7.62 (s, 1H), 7.61-7.59 (m, 1H), 7.48 (d, J=8.1 Hz, 1H), 7.27 (td, J=8.3, 7.8, 1.3 Hz, 1H), 7.22 (td, J=7.5, 0.9 Hz, 1H), 4.46 (t, J=4.6 Hz, 1H), 4.42 (d, J=4.1 Hz, 1H), 3.16 (dd, J=8.0, 6.0 Hz, 1H), 2.86 (dd, J=14.8, 5.9 Hz, 1H), 2.75 (dd, J=14.9, 8.2 Hz, 1H), 2.44 (dd, J=9.0, 4.9 Hz, 1H), 1.80-1.73 (m, 1H), 1.58 (dd, J=11.8, 9.1 Hz, 1H), 1.54-1.34 (m, 4H).

### Example 2: Hydrate Form NF2

### a) Small Scale: Slurry in Aqueous Media

Approximately 15 mg drug substance were suspended in 300 μL of the respective aqueous medium. The suspension was stirred for 5 days at room temperature. Solid/liquid separation was done by centrifugation using 2 mL PE vials and decantation of the centrate. The sample was dried at room temperature and a dry nitrogen flow. As aqueous media Water, Simulated Gastric Fluid (SGF, pH 1.2), Simulated Intestinal Fluid (SIF, pH 6.8) and physiological sodium chloride solution (0.9% m/m NaCl) were used.

### b) Small Scale: Slurry in Water

Approximately 191 mg drug substance was suspended in 2.7 mL water and stirred for 5 days at 50° C. Solid/liquid separation was done by centrifugation using 5 mL PE vials and decantation of the centrate. The sample was dried at room temperature and a dry nitrogen flow.

¹H NMR (700 MHz, Chloroform-d) δ 7.59 (d, J=7.6 Hz, 1H), 7.48 (s, 1H), 7.41 (d, J=8.2 Hz, 1H), 7.24 (t, J=7.4 Hz, 1H), 7.19 (t, J=7.4 Hz, 1H), 6.89 (s, 1H), 4.60 (d, J=4.9 Hz, 1H), 4.56 (t, J=4.9 Hz, 1H), 3.15-3.09 (m, 1H), 2.99 (dd, J=15.1, 5.1 Hz, 1H), 2.81 (dd, J=15.1, 8.8 Hz, 1H), 2.66 (dd, J=9.0, 4.1 Hz, 1H), 1.82 (dd, J=12.4, 9.2 Hz, 1H), 1.76-1.66 (m, 3H), 1.52-1.43 (m, 2H).

c) Small Scale: Cooling Crystallization from Binary Mixtures with Water

Approximately 15 mg drug substance were dissolved in the respective organic solvent: Water mixture at 50° C. and filtered using a 0.2 μm PTFE syringe filter. The solution was cooled by stirring to 5° C. (20° C. in case of DMSO) at 0.1 K/min. Solid/liquid separation of obtained solid material was done by centrifugation using 2 mL PE vials and decantation of the centrate. The sample was dried at room temperature using a dry nitrogen flow. Following compositions were used for the cooling crystallisations.

| | | |
|---|---|---|
| Solvent | Volume | Volume Water |

| | | | | |
|---|---|---|---|---|
| Acetonitrile | 200 | μL | 200 | μL |
| Dimethyl sulfoxide | 1500 | μL | 1000 | μL |
| Dimethylacetamide | 190 | μL | 1000 | μL |

d) Small Scale: Evaporation Crystallization from Binary Mixture with Water

Approx. 15 mg drug substance were dissolved in a mixture of 1 ml Water and 1 ml Acetonitrile at room temperature. The solution was filtered using a 0.2 μm PTFE syringe filter in a new 4 ml glass vial. This glass vial containing the clear solution, was open placed at ambient conditions until the solvent mixture was completely evaporated.

### Example 3: Anhydrous Form NF9

Small Scale: Evaporation Crystallization from Dichloromethane (DCM)

Approx. 50 mg drug substance were dissolved in 500 μL dichloromethane. The solution was filtered using a 0.2 μm PTFE syringe filter in a new 4 ml glass vial. This glass vial containing the clear solution was open placed at ambient conditions or 50° C. until the solvent was completely evaporated.

¹H NMR (500 MHz, DMSO-d6) δ 8.64 (q, J=3.6 Hz, 1H), 7.69 (s, 1H), 7.55 (d, J=7.3 Hz, 1H), 7.52 (d, J=8.1 Hz, 1H), 7.30 (td, J=8.3, 7.9, 1.2 Hz, 1H), 7.24 (td, J=7.6, 0.8 Hz, 1H), 4.58 (t, J=4.8 Hz, 2H), 2.92-2.85 (m, 1H), 2.81 (dd, J=15.2, 6.1 Hz, 1H), 2.71-2.60 (m, 2H), 1.87 (dt, J=10.3, 4.6 Hz, 1H), 1.71 (dd, J=11.9, 9.1 Hz, 1H), 1.65-1.40 (m, 4H)

b) Small Scale: Evaporation Crystallization from Binary Mixtures with DCM

Approximately 16 mg drug substance were dissolved in the respective mixture of an organic solvent with dichloromethane. The solution was filtered using a 0.2 μm PTFE syringe filter in a new 4 mL glass vial. This glass vial containing the clear solution was open placed at ambient conditions until the solvent was completely evaporated. Following combinations were used as binary solvent mixture.

| Solvent | Volume | Volume DCM | |
|---|---|---|---|
| Methyl tert butyl ether | 1 mL | 2 | mL |
| n-Hexane | 1 mL | 1.5 | mL |

### c) Small Scale: Slurry in Tetrahydrofuran (Stabilized by BHT)

Approximately 16-20 mg drug substance were suspended in 0.5 ml Tetrahydrofuran and stirred overnight at ambient conditions or 50° C. Solid/liquid separation was done by centrifugation using 2 mL PE vials and decantation of the centrate. The sample was dried at room temperature and a dry nitrogen flow.

### Example 4: Boronic Acid Ester Form NF3

### Small Scale: Slurry in Isobutanol

A quantity of 15 mg of drug substance was suspended in isobutanol. The suspension was stirred overnight at room temperature. A second suspension was made using 25 mg drug substance in 0.7 mL isobutanol and stirred overnight at 50° C. Solid/liquid separation was done by centrifugation using 2 mL PE vials and decantation of the centrate. The sample was dried at room temperature and a dry nitrogen flow.

¹H NMR (500 MHz, Chloroform-d) δ 7.54-7.48 (m, 1H), 7.40-7.36 (m, 2H), 7.21 (ddd, J=8.3, 7.2, 1.3 Hz, 1H), 7.16 (td, J=7.5, 1.0 Hz, 1H), 6.53-6.43 (m, 1H), 4.52 (t, J=5.5 Hz, 2H), 3.40-3.34 (m, 4H), 3.26-3.18 (m, 1H), 2.98-2.87 (m, 1H), 2.76 (dd, J=14.8, 8.7 Hz, 1H), 2.52 (dd, J=9.1, 4.3 Hz, 1H), 1.79 (dd, J=12.4, 9.1 Hz, 1H), 1.75-1.60 (m, SH), 1.43-1.34 (m, 2H), 0.78 (dd, J=6.7, 1.3 Hz, 12H)

### Example 5: Boronic Acid Ester Form NF4

Small Scale: Slurry in n-Butanol

Approximately 25 mg of drug substance was suspended in 500 μl n-Butanol. The suspension was stirred overnight at 50° C. Solid/liquid separation was done by centrifugation using 2 mL PE vials and decantation of the centrate. The sample was dried at room temperature and a dry nitrogen flow.

### Example 6: Boronic Acid Ester NF5

### Small Scale: Slurry in 2-Propanol

Approximately 62 mg of drug substance were suspended in 2 mL 2-propanol. The suspension was stirred for 5 days at 50° C. Solid/liquid separation was done by centrifugation using 2 mL PE vials and decantation of the centrate. The sample was dried at 50° C. and a dry nitrogen flow for 2 days.

1H NMR (700 MHz, Chloroform-ct) d 7.66 (d, J=7.6 Hz, 1H), 7.57 (s, 1H), 7.50 (d, J=8.2 Hz, 1H), 7.33 (t, J=7.7 Hz, 1H), 7.27 (t, J=7.3 Hz, 1H), 7.13 (s, 1H), 4.66-4.60 (m, 2H), 3.18 (dd, J=15.1, 4.2 Hz, 1H), 3.09 (dd, J=11.0, 4.2 Hz, 1H), 2.93-2.83 (m, 2H), 1.93 (dd, J=12.6, 9.2 Hz, 1H), 1.86-1.70 (m, 3H), 1.57-1.48 (m, 2H) Example 7: Boronic Acid Ester Form NF6

### Small Scale: Slurry in Methanol

Approximately 570 mg of drug substance were suspended in 4 mL Methanol. The suspension was stirred for 5 days at room temperature. Solid/liquid separation was done by centrifugation using 5 mL PE vials and decantation of the centrate. The sample was dried at 50° C. and a dry nitrogen flow for 2 days.

### Example 8: Boronic Acid Adduct Form NF7

a) Small Scale: Cooling Crystallization from Binary Mixtures with Pyridine

Approx. 14-15 mg drug substance was dissolved in the respective solvent: Pyridine mixture at 50° C. and filtered using a 0.2 μm PTFE syringe filter. The solution was cooled by stirring to 5° C. at 0.1 K/min. Solid/liquid separation of obtained solid material was done by centrifugation using 2 mL PE vials and decantation of the centrate. The sample was dried at room temperature using a dry nitrogen flow. The following compositions were used for the cooling crystallisations:

| Solvent | Volume | Volume Pyridine | |
|---|---|---|---|
| Water | 1 mL | 120 | μL |
| Methyl Acetate | 1 mL | 250 | μL |
| Ethyl Acetate | 1 mL | 300 | μL |
| Methyl Isobutyl Ketone | 1 mL | 320 | mL |
| Methyl Isobutyl Ketone | 1 mL | 500 | mL |
| Ethyl Acetate | 1 mL | 300 | μL |

¹H NMR (500 MHz, Chloroform-ct) d 8.62 (dd, J=5.8, 1.6 Hz, 0.67H), 7.67 (tt, J=7.6, 1.8 Hz, 0.33H), 7.59 (d, J=7.1 Hz, 1H), 7.48 (s, 1H), 7.41 (d, J=8.1 Hz, 1H), 7.30-7.27 (m, 0.67H), 7.23 (dd, J=8.1, 1.1 Hz, 1H), 7.21-7.14 (m, 1H), 6.90-6.83 (m, 1H), 4.60 (d, J=4.9 Hz, 1H), 4.55 (t, J=5.0 Hz, 1H), 3.12 (ddd, J=8.3, 5.3, 2.9 Hz, 1H), 2.99 (dd, J=15.2, 4.9 Hz, 1H), 2.81 (dd, J=15.1, 8.7 Hz, 1H), 2.66 (dd, J=9.0, 4.2 Hz, 1H), 1.82 (dd, J=12.5, 9.1 Hz, 1H), 1.78-1.60 (m, 3H), 1.51-1.42 (m, 2H)

b) Small Scale: Evaporation Crystallization from Pyridine

Approximately 14 mg drug substance were dissolved in 3 mL pyridine. The solution was filtered using a 0.2 μm PTFE syringe filter in a new 4 mL glass vial. This glass vial containing the clear solution was open placed at ambient conditions until the solvent was completely evaporated.

c) Small Scale: Anti-Solvent Precipitation from Pyridine

1 mL anti-solvent was added by stirring to a 100 μl aliquot from a Pyridine stock solution (301 mg dissolved in 1 mL). The mixture was stirred at room temperature until a precipitation occurred. Solid/liquid separation of obtained solid material was done by centrifugation using 2 mL PE vials and decantation of the centrate. The sample was dried at room temperature using a dry nitrogen flow. Following anti-solvents were used for the precipitation experiments: water, acetone, methyl ethyl ketone, and n-heptane.

### Example 9: Boronic Acid Ester NF8

### Small Scale: Slurry in 1-Propanol

More than 75 mg of drug substance was suspended in 1 mL 1-propanol. The suspension was stirred overnight at room temperature. Solid/liquid separation was done by centrifugation using 5 mL PE vials and decantation of the centrate. The sample was dried at room temperature and a dry nitrogen flow.

### Example 10: Solubility Data of Forms A1 and NF2

### Thermodynamic Solubility Data at 37° C.

Approximately 10-20 mg of 1-(4-{[6-Amino-5-(4-phenoxy-phenyl)-pyrimidin-4-ylamino]-methyl}-4-fluoro-piperidin-1-yl)-propenone was weighed into a 4 mL glass vial. 1 mL of SGF medium (simulated gastric fluid, pH 1.2), FaSSIF medium (fasted state intestinal medium, pH 6.5), or USP Phosphate buffer pH 7.4 was added and the suspension was shaken for 24 h at 450 rpm at 37° C. After 1 hour, 6 hours and after 24 hours, the vials were checked for the presence of undissolved compound and the pH was measured. If necessary, the pH was adjusted after 1 hour and 6 hours. The solid liquid separation was carried out using 1 mL syringe and 0.2 μm syringe filter. Clear filtrate was analyzed by HPlC after suitable dilution to measure the amount of API dissolved.

Results from thermodynamic solubility determinations are summarised below.

| | Thermodynamic | Thermodynamic | Thermodynamic |
|---|---|---|---|
| | solubility | solubility | solubility PBS |
| Form | SGF pH 1.2 | FaSSIF pH 6.5 | buffer pH 7.4 |
| Trimeric | 2.3 mg/mL | 2.0 mg/mL | 2.0 mg/mL |
| form A1 | | | |
| Form NF2 | 0.7 mg/mL | 0.7 mg/mL | Not |
| | | | determined |

### Example 11: Mini-Dissolution Data of Trimeric Adduct Form A1

Approximately 4 mg of drug substance was weighed into a 12 mL glass vials. 8 mL of SGF medium (simulated gastric fluid, pH 1.2, prewarmed to 37° C.) was added and the suspension was shaken at 450 rpm at 37° C. After 30 min, 60 min, and 120 min, 1 mL of the suspension was withdrawn and filtered through a 0.2 μm syringe filter. The clear filtrate was analyzed by HPLC after suitable dilution to measure the amount of API dissolved.

Results from the mini-dissolution studies are summarized below:

| | Dissolution |
|---|---|
| | Levels in SGF |
| Time | (pH 1.2) |
| 30 minutes | 1.9 mg/mL |
| 60 minutes | 2.2 mg/mL |
| 120 minutes | 2.4 mg/mL |

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

1. A solid form of compound 1, 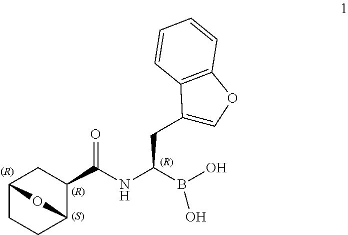
or a pharmaceutically acceptable salt, hydrate, solvate, ester or adduct thereof.

2. The solid form of claim 1, wherein the solid form is a trimeric crystalline anhydrous form A1.

3. The solid form of claim 2, wherein the trimeric crystalline anhydrous form A1 is characterized by two or more 2θ XRPD peaks at 6.5±0.2°, 11.2±0.2°, 17.1±0.2°, 19.6±0.2°, and 21.9±0.2° degrees.

4. The solid form of claim 2, wherein the trimeric crystalline anhydrous form A1 is characterized by four or more 2θ XRPD peaks at 6.5±0.2°, 8.6±0.2°, 11.2±0.2°, 13.8±0.2°, 14.1±0.2°, 15.2±0.2°, 16.3±0.2°, 17.1±0.2°, 18.2±0.2°, 18.9±0.2°, 19.6±0.2°, 20.3±0.2°, 20.7±0.2°, 21.4±0.2°, 21.9±0.2°, 22.5±0.2°, and 25.7±0.2° degrees.

5. The solid form of claim 2, wherein the trimeric crystalline anhydrous form A1, comprises an orthorhombic space group P-2₁2₁2₁ with the lattice parameters a=10.99±0.1 Å, b=15.80±0.1 Å, c=27.27±0.1 Å, and α=, β=γ=90.0±0.1°.

6. The solid form of claim 1, wherein the solid form is a crystalline boronic acid ester form NF6, wherein the crystalline boronic acid ester form NF6 is characterized by two or more 2θ XRPD peaks at 12.5±0.2°, 15.0±0.2°, 18.0±0.2°, 20.6±0.2°, and 21.3±0.2° degrees.

7. The solid form of claim 6, wherein the crystalline boronic acid ester form NF6 is characterized by four or more 2θ XRPD peaks at 10.3±0.2°, 12.5±0.2°, 15.0±0.2°, 15.9±0.2°, 18.0±0.2°, 19.3±0.2°, 20.1±0.2°, 20.6±0.2°, 21.3±0.2°, 22.0±0.2°, 24.0±0.2°, 24.6±0.2°, 25.2±0.2°, 25.8±0.2°, 27.9±0.2°, and 29.5±0.2° degrees.

8. (canceled)

9. The solid form of claim 1, wherein the solid form is at least one selected from the group consisting of the solvate form NF2 of compound 1, anhydrous form NF9 of compound 1, boronic acid ester form NF3 of compound 1, boronic acid ester form NF4 of compound 1, boronic acid ester form NF5 of compound 1, hydrate form NF2 of compound 1, hydrate of a trimeric adduct form NF7 of compound 1, and boronic acid adduct form NF8 of compound 1.

10. The anhydrous form NF9 of compound 1 of claim 9, wherein the anhydrous form NF9 of compound 1 is characterized by two or more 2θ XRPD peaks at 6.5±0.2°, 18.0±0.2°, 19.5±0.2°, and 20.7±0.2° degrees.

11. The boronic acid ester form NF3 of compound 1 of claim 9, wherein the boronic acid ester form NF3 of compound 1 is characterized by two or more 2θ XRPD peaks at 7.8±0.2°, 12.5±0.2°, 17.1±0.2°, 20.6±0.2°, 21.2±0.2°, and 22.0±0.2° degrees.

12. The boronic acid ester form NF4 of compound 1 of claim 9, wherein the boronic acid ester form NF4 of compound 1 is characterized by two or more 2θ XRPD peaks at 7.4±0.2°, 8.0±0.2°, 18.0±0.2°, 18.7±0.2°, and 22.2±0.2° degrees.

13. The boronic acid ester form NF5 of compound 1 of claim 9, wherein the boronic acid ester form NF4 of compound 1 is characterized by two or more 2θ XRPD peaks at 5.8±0.2°, 18.4±0.2°, 18.7±0.2°, 19.0±0.2°, and 21.7±0.2° degrees.

14. The hydrate form NF2 of compound 1 of claim 9, wherein the hydrate form NF2 of compound 1 is characterized by two or more 2θ XRPD peaks at 7.0±0.2°, 16.0±0.2°, 18.1±0.2°, 19.5±0.2°, and 19.9±0.2° degrees.

15. The hydrate of a trimeric adduct form NF7 of compound 1 of claim 9, wherein the hydrate of the trimeric adduct form NF7 of compound 1 is characterized by two or more 2θ XRPD peaks at 10.5±0.2°, 17.2±0.2°, 18.1±0.2°, and 21.7±0.2° degrees.

16. The solid form of claim 15, wherein a crystalline hydrate of the trimeric adduct form NF7 of compound 1 is characterized by four or more 2θ XRPD peaks at 8.3±0.2°, 8.6±0.2°, 10.5±0.2°, 12.8 0.2°, 16.0±0.2°, 17.2±0.2°, 18.1±0.2°, 19.2±0.2°, 19.8±0.2°, 21.0±0.2°, 21.7±0.2°, 22.6±0.2°, and 25.5±0.2° degrees.

17. The solid form of claim 15, wherein the crystalline hydrate of the trimeric adduct form NF7 of compound 1 comprises a monoclinic space group P-2₁ with the lattice parameters a=14.02±0.1 Å, b=9.18±0.1 Å, c=20.84±0.1 Å, and α=γ=90.0±0.1°, and β=99.58±0.1°.

18. The boronic acid adduct form NF8 of compound 1 of claim 9, wherein the boronic acid adduct form NF8 of compound 1 is characterized by two or more 2θ XRPD peaks at 9.41±0.2°, 10.3±0.2°, 15.8±0.2°, and 17.5±0.2° degrees.

19. A pharmaceutical composition, comprising
a solid form of compound 1 of claim 1, and a pharmaceutically acceptable adjuvant, carrier, or vehicle.

20. A method for treating a LMP7-mediated disorder in a patient in need thereof, the method comprising:
administering to said patient a solid form of compound 1 of claim 1.

21. (canceled)

22. (canceled)

23. A method for producing a medicament for the prophylactic or therapeutic treatment of a LMP7-mediated disorder, the method comprising:
formulating a solid form of compound 1 of claim 1 or a pharmaceutically acceptable salt thereof.

24. (canceled)

25. (canceled)
